(19) United States
(12) Patent Application Publication
(10) Pub. No.: US 2023/0165836 A1
(43) Pub. Date: Jun. 1, 2023

(54) ALPHA-2 ADRENERGIC RECEPTOR AGONISTS FOR THE TREATMENT OF CANCER

(71) Applicant: UNIVERSITÉ CATHOLIQUE DE LOUVAIN, Louvain-la-Neuve (BE)

(72) Inventors: Benoît VAN DEN EYNDE, Genval (BE); Jingjing ZHU, Zaventem (BE)

(73) Assignee: UNIVERSITÉ CATHOLIQUE DE LOUVAIN, Louvain-la-Neuve (BE)

(21) Appl. No.: 17/920,116

(22) PCT Filed: Apr. 21, 2021
(86) PCT No.: PCT/EP2021/060356

§ 371 Date: Oct. 20, 2022

(30) Foreign Application Priority Data
Apr. 21, 2020 (EP) 20170702.3

(51) Int. Cl. A61K 31/4168; A61P 17/00; A61K 31/155; A61P 1/00; A61P 15/00; A61K 31/165; A61P 35/02; A61P 11/00; A61P 35/00; A61P 1/16; A61P 13/12

(52) U.S. Cl. CPC A61K 31/4168; A61P 17/00; A61K 31/155; A61P 1/00; A61P 15/00; A61K 31/165; A61P 35/02; A61P 11/00; A61P 35/00; A61P 1/16; A61P 13/12

(57) ABSTRACT

The present invention relates to the treatment of cancer. In particular, the invention relates to the therapeutic use of alpha-2 adrenergic receptor agonists for the treatment of cancer. More particularly, apraclonidine, clonidine, guanfacine and guanabenz, which are alpha-2 adrenergic receptor agonists, are all capable of efficiently reducing the growth of solid tumors. The effect is immune-mediated and is abolished in the presence of an alpha-2 antagonist or in mice that are knockout for the alpha-2 adrenergic receptor.

### FIELD OF INVENTION

The present invention relates to the prevention and/or treatment of cancer. In particular, the invention relates to the therapeutic use of alpha-2 adrenergic receptor agonists for the treatment of cancer. More particularly, apraclonidine, clonidine, guanfacine and guanabenz, which are alpha-2 adrenergic receptor agonists, are all capable of efficiently reducing the growth of solid tumors, as a monotherapy.

### BACKGROUND OF INVENTION

As for 2018, it can be estimated that there were 17 million new cases of cancer worldwide, and that 9.6 million deaths were directly attributable to cancer. Among the most commonly occurring cancers worldwide, four are solid cancers, namely lung cancer, female breast cancer, bowel cancer and prostate cancer. These four types of cancer account for more than 40% of all cancers diagnosed in the world. Estimations predict that there will be globally around 27.5 million new cases of cancer each year by 2040.

WO2012001065 showed that it was possible to prevent skin tumor formation and to delay the onset of skin tumors, by the mean of administration of a composition comprising an alpha-2 adrenergic receptor agonist.

Upon being diagnosed, the main goals of treating cancer include entirely eradicating diagnosed tumors, preventing the recurrence or spread of the primary cancer, i.e., preventing metastasis, and relieving symptoms if all reasonable curative approaches have been exhausted. However, medical decisions concerning how to treat a particular cancer are relying upon many factors.

Surgery, radiation-based surgical knives, chemotherapy, and radiotherapy are some of the traditional and most widely used treatment to cure cancer. In most of the cases, an average of 50% of patients diagnosed with cancer undergo surgery, as a primary treatment, so as to remove the tumor. Radiations and chemotherapy represent each alternative or secondary treatments to surgery.

Modern approaches are being developed, such as, e.g., hormone-based therapy, anti-angiogenic therapies, stem cell therapies, immunotherapy, and dendritic cell-based immunotherapy. In addition, based on individual markers, personalized medicine is currently developed in order to offer a therapy that is adapted to the patient to be treated instead to offering therapies based on statistical data, i.e., evaluating overall chances to succeed. However, these last approaches are time-consuming and request large resources.

To date, a great deal of in vitro approaches to assess the efficacy of therapeutic compounds has been developed as a first step in the provision of cancer treatments. This is illustrated, e.g., by the study of Kanno et al. (Hepatology, 2002, Vol. 35(6), p. 1329-1340), which showed that UK14,304 (brimonidine tartrate) treatment significantly decreases cholangiocarcinoma growth. However, in vitro only experimental data have the drawback of not capturing the involvement of the environment of the cancer cells, in particular the immune cells of the host, which play a major role in the response to cancer treatment.

There is a need to identify new universal anti-cancer compounds. In particular, there is a need to identify compounds that could be administered to treat solid cancer, as an alternative to surgery.

### SUMMARY

One aspect of the invention relates to an alpha-2 adrenergic receptor agonist as an active agent for use in the treatment of cancer.

In certain embodiments, said agonist is selected in the group consisting of amitraz, apraclonidine, bethanidine, brimonidine, bromocriptine, cirazoline, clonidine, detomidine, dexmedetomidine, dipivefrin, droxidopa, epinephrine, ergotamine, etilefrine, etomidate, fadolmidine, guanabenz, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof. In some embodiments, said agonist is selected in the group consisting of apraclonidine, clonidine, guanfacine, romifidine, and a functional derivative thereof. In certain embodiments, the alpha-2 adrenergic receptor agonist is selected in the group consisting of an antibody, an antibody fragment, an afucosylated antibody, a diabody, a triabody, a tetrabody, a nanobody, and an analog thereof. In some embodiments, said agonist does not cross the blood/brain barrier. In some embodiments, said agonist is to be administered at a dose ranging from about 0.0001 mg/kg body weight to about 100 mg/kg body weight. In certain embodiments, said agonist is to be administered systemically. In some embodiments, said agonist is to be administered with an additional treatment selected in the group consisting of chemotherapy, immunotherapy, radiation, and the like. In certain embodiments, said cancer is selected in the group consisting of myelofibrosis, acute lymphoblastic leukemia, acute myeloblastic leukemia adrenal gland carcinoma, bile duct cancer, bladder cancer, breast cancer, cervical cancer, colorectal cancer, endometrial cancer, esophageal cancer, gastric cancer, gastrointestinal stromal tumors, glioblastoma, head and neck cancer, hepatocellular carcinoma, Hodgkin's lymphoma, kidney cancer, lung cancer, melanoma, Merkel cell skin cancer, mesothelioma, multiple myeloma, myeloproliferative disorders, non-Hodgkin lymphoma, ovarian cancer, pancreatic cancer, prostate cancer, salivary gland cancer, sarcoma, squamous cell carcinoma, testicular cancer, thyroid cancer, urothelial carcinoma, and uveal melanoma. In certain embodiments, the cancer is a solid cancer. In some embodiments, the solid cancer is selected in the group consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, fibrosarcoma, glioblastoma, prostate carcinoma, ovarian cancer and pancreatic carcinoma. In one embodiment, the solid cancer is selected in the group consisting of colon carcinoma, ovarian cancer, melanoma, breast carcinoma, liver carcinoma, lung carcinoma, renal carcinoma, prostate carcinoma and fibrosarcoma.

In certain embodiments, the agonist is for use for reducing the volume and/or the weight of a solid tumor.

Another aspect of the invention pertains to a pharmaceutical composition comprising an alpha-2 adrenergic receptor agonist, as defined herein, and a pharmaceutically acceptable carrier, for use in the treatment of cancer.

In another aspect, the invention relates to a method for the treatment of cancer in an individual in need thereof comprising the administration of a therapeutic effective amount of an alpha-2 adrenergic receptor agonist, as an active agent.

### DEFINITIONS

In the present invention, the following terms have the following meanings:
“About” preceding a figure encompasses plus or minus 10%, or less, of the value of said figure. It is to be understood that the value to which the term “about” refers to is itself also specifically, and preferably, disclosed.
“Comprise” is intended to mean “contain”, “encompass” and “include”. In some embodiments, the term “comprise” also encompasses the term “consist of”.
“Alpha-2 adrenergic receptor agonist” refers to a compound capable of activating alpha-2 adrenergic receptors, upon binding to those receptors. Within the scope of the instant invention, the term “activating alpha-2 adrenergic receptors” is intended to mean that upon activation, these receptors are capable of inhibiting norepinephrine release from presynaptic neurons, and/or centrally inducing sedation via locus coeruleus, and/or inhibiting insulin release from pancreatic 13 cells.
“Functional derivative”, when referred to the alpha-2 adrenergic receptor agonist according to the invention, is intended to refer to a derivate of an alpha-2 adrenergic receptor agonist that shares an equivalent biological physiological function, while having a similar structure. The term “having a similar structure” is intended to mean that the derivate of the alpha-2 adrenergic receptor agonist differs from the reference alpha-2 adrenergic receptor agonist in that it possesses one or more substituent(s).
“Active agent” is intended to mean that the alpha-2 adrenergic receptor agonist according to the invention represents the therapeutic compound, which promotes, by itself, a suitable prophylactic or therapeutic cancer treatment.
“Monotherapy” refers to the alpha-2 adrenergic receptor agonist according to the invention representing the sole therapeutic compound for the use in the prevention and/or treatment of cancer.
“Treating” or “treatment” or “alleviation” refers to both therapeutic treatment and prophylactic or preventative measures, wherein the object is to prevent or slow down (lessen) the targeted pathologic condition or disorder, in particular cancer. Those in need of treatment include those already with said disorder as well as those prone to develop the disorder or those in whom the disorder is to be prevented. An individual is successfully “treated” for cancer if, after receiving a therapeutic amount of the alpha-2 adrenergic receptor agonist according to the present invention, the individual shows observable and/or measurable reduction in or absence of one or more of the symptoms associated with cancer; reduced morbidity and mortality, and improvement in quality of life issues. The above parameters for assessing successful treatment and improvement in the disease are readily measurable by routine procedures familiar to physician or authorized personnel.
“Preventing” refers to keeping from happening, and/or lowering the chance of the onset of, or at least one adverse effect or symptom of, cancer, disorder or condition associated with a deficiency in or absence of an organ, tissue or cell function.
“Efficient amount” refers to the level or the amount of the active agent that is aimed at, without causing significant negative or adverse side effects to the target, (1) delaying or preventing the onset of cancer; (2) slowing down or stopping the progression, aggravation, or deterioration of one or more symptoms of cancer; (3) bringing about ameliorations of the symptoms of cancer; (4) reducing the severity or incidence of cancer; or (5) curing cancer. A therapeutically effective amount may be administered prior to the onset of cancer, for a prophylactic or preventive action. Alternatively, or additionally, the therapeutically effective amount may be administered after the onset of cancer, for a therapeutic action. In one embodiment, a therapeutically effective amount of the composition is an amount that is effective in reducing at least one symptom of cancer.
“Pharmaceutically acceptable carrier” refers to a carrier, or vehicle, that does not produce any adverse, allergic or other unwanted reactions when administered to an animal individual, preferably a human individual. It includes any and all solvents, dispersion media, coatings, antibacterial and antifungal agents, isotonic and absorption delaying agents and the like. For human administration, preparations should meet sterility, pyrogenicity, general safety, quality and purity standards as required by regulatory Offices, such as, e.g., the FDA in the United States or the EMA in the European Union.
“Individual” is intended to refer to an animal individual, preferably a mammalian individual, more preferably a human individual. Among the non-human mammalian individuals of interest, one may non-limitatively mention pets, such as dogs, cats, guinea pigs; animals of economic importance such as cattle, sheep, goats, horses, monkeys. In one embodiment, an individual may be a “patient”, i.e. a warm-blooded animal, more preferably a human, who/which is awaiting the receipt of, or is receiving medical care or was/is/will be the object of a medical procedure, or is monitored for the development of a disease, disorder or condition. In one embodiment, the individual is an adult (for example a human subject above the age of 18). In another embodiment, the individual is a child (for example a human subject below the age of 18). In one embodiment, the individual is a male. In another embodiment, the individual is a female.

### DETAILED DESCRIPTION

The inventors previously observed that guanabenz, an alpha-2 adrenergic receptor agonist, is an adjuvant of immunotherapy for the treatment of cancer (WO2020083982). Guanabenz has been wrongly described as having the capacity of inhibiting STATS activity (WO2008156644).

Surprisingly, by the means of in vivo experimental data obtained with several models, the inventors showed herein that alpha-2 adrenergic receptor agonists can be used as a monotherapy for the treatment of cancer. More particularly, it is the agonistic mechanism that is important to observe the anti-cancer effect, as demonstrated by the absence of any anti-cancer properties in the presence of an antagonist of the alpha-2 adrenergic receptor or in an alpha-2 adrenergic receptor knock-out mice. The inventors hence demonstrated that the structure of the alpha-2 adrenergic receptor agonists is not at stake to explain the benefit towards cancer treatment but rather their function. In addition, the inventors herein provided evidences that the anti-tumor properties of alpha-2 adrenergic receptor agonists are applicable to various types of cancers, such as blood cancers and solid cancers.

One aspect of the invention relates to an alpha-2 adrenergic receptor agonist as an active agent for use in the prevention and/or the treatment of cancer.

A further aspect of the invention pertains to an alpha-2 adrenergic receptor agonist as an active agent for use in the treatment of cancer.

In some aspect, the invention further relates to the use of an alpha-2 adrenergic receptor agonist as an active agent for the prevention and/or the treatment of cancer. The invention also relates to the use of an alpha-2 adrenergic receptor agonist as an active agent for the treatment of cancer.

In practice, the cancer may be diagnosed by any suitable method known in the state of the art, or method adapted therefrom. Non-limitative examples of suitable methods include biopsies; blood tests, such as a complete blood count (numbers of red blood cells, white blood cells and platelets); ultrasound or computerized tomography (CT) scan; magnetic resonance imagining (MRI); endoscopy; dosing tumoral markers; and the like.

In some embodiments, the alpha-2 adrenergic receptor agonist is a small organic molecule, a peptide, a polypeptide or a protein.

As used herein, the term “small organic molecule” is intended to refer to an organic molecule that has a molar weight inferior to about 1,000 g/mol, preferably inferior to about 750 g/mol, more preferably inferior to about 600 g/mol. Within the scope of the invention, the expression “inferior about 1,000 g/mol” encompasses inferior to about 1,000 g/mol, 950 g/mol, 900 g/mol, 850 g/mol, 800 g/mol, 750 g/mol, 700 g/mol, 650 g/mol, 600 g/mol, 550 g/mol, 500 g/mol, 450 g/mol, 400 g/mol, 350 g/mol, 300 g/mol, 250 g/mol and 200 g/mol. In practice, the molar weight of a molecule may be determined by any suitable methods acknowledged in the state of the art, or a method adapted therefrom. Non-limitative examples of suitable methods include mass spectrometry, nuclear magnetic resonance (NMR), and the like.

As used herein, the term “peptide” refers to a linear polymer of amino acids of less than 50 amino acid residues linked together by peptide bonds; the term “polypeptide” refers to a linear polymer of at least 50 amino acid residues linked together by peptide bonds; and the term “protein” specifically refers to a functional entity formed of one or more peptides or polypeptides

In some embodiments, the alpha-2 adrenergic receptor agonist is a small organic molecule.

In certain embodiments, said agonist is selected in the group consisting of amitraz, apraclonidine, bethanidine, brimonidine, bromocriptine, cirazoline, clonidine, detomidine, dexmedetomidine, dipivefrin, droxidopa, epinephrine, ergotamine, etilefrine, etomidate, fadolmidine, guanabenz, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof.

As defined herein, it is to be understood that amitraz, apraclonidine, bethanidine, brimonidine, bromocriptine, cirazoline, clonidine, detomidine, dexmedetomidine, dipivefrin, droxidopa, epinephrine, ergotamine, etilefrine, etomidate, fadolmidine, guanabenz, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof, are small organic molecules.

In some embodiments, said agonist is selected in the group consisting of amitraz, apraclonidine, bethanidine, bromocriptine, cirazoline, clonidine, detomidine, dexmedetomidine, dipivefrin, droxidopa, epinephrine, ergotamine, etilefrine, etomidate, fadolmidine, guanabenz, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof.

In certain embodiments, said agonist is selected in the group consisting of amitraz, apraclonidine, bethanidine, brimonidine, bromocriptine, cirazoline, clonidine, detomidine, dexmedetomidine, dipivefrin, droxidopa, epinephrine, ergotamine, etilefrine, etomidate, fadolmidine, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof.

In certain embodiments, said agonist is selected in the group consisting of amitraz, apraclonidine, bethanidine, bromocriptine, cirazoline, clonidine, detomidine, dexmedetomidine, dipivefrin, droxidopa, epinephrine, ergotamine, etilefrine, etomidate, fadolmidine, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof.

In some embodiments, said agonist is selected in the group consisting of apraclonidine, brimonidine, clonidine, dexmedetomidine, guanabenz, guanfacine, lofexidine, methyldopa, romifidine, tizanidine, xylazine, and a functional derivative thereof.

In some embodiments, said agonist is selected in the group consisting of apraclonidine, clonidine, guanabenz, guanfacine, romifidine, and a functional derivative thereof.

In certain embodiments, the alpha-2 adrenergic receptor agonist is not guanabenz, or a functional derivative thereof. In some embodiments, said agonist is selected in the group consisting of apraclonidine, brimonidine, clonidine, dexmedetomidine, guanfacine, lofexidine, methyldopa, romifidine, tizanidine, xylazine, and a functional derivative thereof.

In some embodiments, said agonist is selected in the group consisting of apraclonidine, clonidine, guanfacine, romifidine, and a functional derivative thereof.

In certain embodiments, the alpha-2 adrenergic receptor agonist is not brimonidine, or a functional derivative thereof. In some embodiments, said agonist is selected in the group consisting of apraclonidine, clonidine, dexmedetomidine, guanabenz, guanfacine, lofexidine, methyldopa, romifidine, tizanidine, xylazine, and a functional derivative thereof.

In some embodiments, said agonist is selected in the group consisting of apraclonidine, clonidine, dexmedetomidine, guanfacine, lofexidine, methyldopa, romifidine, tizanidine, xylazine, and a functional derivative thereof.

In certain embodiments, said agonist is apraclonidine, clonidine, romifidine, or a functional derivative thereof.

As used herein, a functional derivative of an alpha-2 adrenergic receptor agonist according to the invention is an agonist that possesses one or more substituent(s), in particular a substituent selected in the group consisting of a linear or ramified group; an alkyl group, an aryl group, an amine group, an amide group, a sulfide group, a bromide group, a chloride group, a fluoride group, carbonyl (C═O) group, formyl group, hydroxyl group, aldehyde group, alkoxy (O—R) group. Other groups further include: alkenyl, alkynyl, carboxamide, primary amine, R2NH, R3N, R4N+, azide, azo(diimide), benzyl, carbonate ester, carboxylate, carboxyl, cyanate, RSCN, disulfide, ether, ester, hydroperoxy, primary ketimine, RC(═NR)R′, RC(═NH)H, RC(═NR′)H, imide, isocyanide, isocyanate, RNCS, nitrate, nitrile, nitrosooxy, nitro, nitroso, peroxy, phenyl, phosphino,phosphate, phosphono, pyridyl, sulfonyl, sulfo, sulfinyl, sulfhydryl, fluoro, chloro, bromo, iodo, haloformyl, carboalkoxy, hemiacetal, hemiketal, acetal, ketal, orthoester, methylenedioxy, orthocarbonate ester, carboxylic anhydride, imine, azo(diimide), isonitrile, oxime, carbamate, sulfide, sulfino, thiocyanate, isothiocyanate, carbonothioyl, carbothioic o-acid, thiolester, thionoester, carbodithioic acid, carbodithio, borono, boronate, borino, borinate, alkyllithium, alkylmagnesium halide, alkylaluminium, silyl ether group, and a combination thereof. In practice, R and R′ groups are non-limitatively referring to alkyl, alkenyl, alkynyl groups.

Within the scope of the invention, an alpha-2 adrenergic receptor agonist functional derivative is an agonist of the alpha-2 receptor.

In certain embodiments, the alpha-2 adrenergic receptor agonist is a peptide, a polypeptide or a protein.

In some embodiments, the alpha-2 adrenergic receptor agonist is selected in the group consisting of an antibody, an antibody fragment, an afucosylated antibody, a diabody, a triabody, a tetrabody, a nanobody, and an analog thereof.

As used herein, an “antibody”, also referred to as immunoglobulins (abbreviated “Ig”), is intended to refer to a gamma globulin protein that is found in blood or other bodily fluids of vertebrates, and is often used by the immune system to identify and neutralize foreign objects, such as bacteria and viruses. Antibodies consist of two pairs of polypeptide chains, called heavy chains and light chains that are arranged in a Y-shape. The two tips of the Y are the regions that bind to antigens and deactivate them. The term “antibody” (Ab) as used herein includes monoclonal antibodies, polyclonal antibodies, multi-specific antibodies (e.g., bispecific antibodies), and antibody fragments, so long as they exhibit the desired biological activity. The term “immunoglobulin” (Ig) is used interchangeably with “antibody” herein.

As used herein, an “antibody fragment” comprises a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870; Zapata et al., Protein Eng. 1995; 8(10): 1057-1062); single-chain antibody molecules; and multi-specific antibodies formed from antibody fragments. One may refer to a “functional fragment or analog” of an antibody, which is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc[epsilon]RI. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

As used herein, an “afucosylated antibody” refers to an antibody lacking core fucosylation. As a matter of fact, nearly all IgG-type antibodies are N-glycosylated in their Fc moiety. Typically, a fucose residue is attached to the first N-acetylglucosamine of these complex-type N-glycans. In other words, an “afucosylated antibody” refers to an antibody that does not possess N-glycans.

As used herein, the term “diabody” refers to a small antibody fragment prepared by constructing sFv fragments with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described in more details in, e.g., EP0404097; WO 93/11161; and Hollinger et al., Proc. Natl. Acad. Sci. USA, 1993; 90:6444-6448.

As used herein, a “triabody” is intended to refer to an antibody that has three Fv heads, each consisting of a VH domain from one polypeptide paired with the VL domain from a neighboring polypeptide.

As used herein, a “nanobody” refers to a functional antibody that consists of heavy chains only and therefore lack light chains. These heavy-chain only antibodies contain a single variable domain (VHH) and two constant domains (CH2, CH3).

In certain embodiments, the alpha-2 adrenergic receptor agonist according to the invention does not cross the blood/brain barrier.

In some embodiments, the alpha-2 adrenergic receptor agonist that does not cross the blood/brain barrier is selected in the group consisting of apraclonidine, ST-91 (also referred to as N-(2,6-Diethylphenyl)-4,5-dihydro-1H-imidazol-2-amine hydrochloride), corbadrine (also referred to as 4-(2-amino-1-hydroxy-propyl)benzene-1,2-diol, or α-methylnorepinephrine), and naphazoline.

In certain embodiments, the alpha-2 adrenergic receptor agonist that does not cross the blood/brain barrier is apraclonidine.

In some embodiments, the alpha-2 adrenergic receptor agonist that does not cross the blood/brain barrier is selected in the group consisting of an antibody, an antibody fragment, an afucosylated antibody, a diabody, a triabody, a tetrabody, a nanobody, and an analog thereof.

In practice, assessing whether a compound, e.g., an alpha-2 adrenergic receptor agonist according to the invention, crosses the blood/brain barrier may be performed by any suitable method acknowledged from the state of the art, or a method adapted therefrom. Illustratively, this assessment may be performed by the means of one of the two gold-standard experimental measures of blood/brain barrier permeability, namely, (1) logBB, which is intended to measure the concentration of a compound in the brain divided by concentration in the blood; and (2) logPS, which measures the permeability surface-area product.

In practice, the total daily dose of the agonist according to the invention may be decided by the attending physician within the scope of sound medical judgment. The specific dose for any particular subject will depend upon a variety of factors such as the severity of the cancer to be treated. Illustratively, these factors include age, body weight, general health, gender and diet of the patient, and additional factors well-known in the medical arts.

In certain embodiments, the individual to be treated is a human or a non-human mammal, preferably a human. In some embodiments, the non-human mammal to be treated is selected in a group comprising a pet such as a dog, a cat, a domesticated pig, a rabbit, a ferret, a hamster, a mouse, a rat and the like; a primate such as a chimp, a monkey, and the like; an economically important animal such as cattle, a pig, a rabbit, a horse, a sheep, a goat, a mouse, a rat, and the like.

In some embodiments, said agonist is to be administered at a dose ranging from about 0.0001 mg/kg body weight to about 100 mg/kg body weight.

In certain embodiments, said agonist is to be administered at a dose ranging from about 0.01 mg/kg body weight to about 100 mg/kg body weight.

Within the scope of the instant invention, the term “about 0.0001 mg/kg body weight to about 100 mg/kg body weight” encompasses about 0.0001, 0.0002, 0.0003, 0.0004, 0.0005, 0.0006, 0.0007, 0.0008, 0.0009, 0.001, 0.002, 0.0003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95 and 100 mg/kg body weight.

In certain embodiments, the dose is ranging from about 0.0001 mg/kg body weight to about 30 mg/kg body weight, preferably from about 0.0001 mg/kg body weight to about 15 mg/kg body weight, more preferably from about 0.0001 mg/kg body weight to about 7 mg/kg body weight. In some embodiments, the dose is ranging from about 0.01 mg/kg body weight to about 30 mg/kg body weight, preferably from about 0.01 mg/kg body weight to about 15 mg/kg body weight, more preferably from about 0.01 mg/kg body weight to about 7 mg/kg body weight.

In certain embodiments, the daily dose is ranging from about 0.0001 mg/day/kg body weight to about 100 mg/day/kg body weight, in particular from about 0.0001 mg/day/kg body weight to about 30 mg/day/kg body weight, preferably from about 0.0001 mg/day/kg body weight to about 15 mg/day/kg body weight, more preferably from about 0.0001 mg/day/kg body weight to about 7 mg/day/kg body weight. In some embodiments, the daily dose is ranging from about 0.01 mg/day/kg body weight to about 100 mg/day/kg body weight, in particular from about 0.01 mg/day/kg body weight to about 30 mg/day/kg body weight, preferably from about 0.01 mg/day/kg body weight to about 15 mg/day/kg body weight, more preferably from about 0.01 mg/day/kg body weight to about 7 mg/day/kg body weight.

In certain embodiments, the alpha-2 adrenergic receptor agonist according to the invention may be, or is to be, administered orally, systemically, parenterally, topically, by inhalation spray, rectally, nasally, buccally, vaginally or via an implanted medical device. In some embodiments, the alpha-2 adrenergic receptor agonist according to the invention may be, or is to be, administered systemically.

According to some embodiments, the agonist according to the instant invention is formulated in a suitable form for an oral administration. Thus, in one embodiment, said agonist is to be administered orally to the subject, for example in the form of a powder, a tablet, a capsule, and the like or as a tablet formulated for extended or sustained release. Non-limitative examples of forms suitable for oral administration include, e.g., liquid, paste or solid compositions, and more particularly tablets, tablets formulated for extended or sustained release, capsules, pills, dragees, liquids, gels, syrups, slurries, suspensions, and the like.

In certain embodiments, said agonist is to be administered systemically.

According to another embodiments, the agonist according to the invention is in an adapted form for an injection. Illustratively, said agonist is thus to be injected to the subject, by intravenous, intramuscular, intraperitoneal, intrapleural, subcutaneous, transdermal injection or infusion. Sterile injectable forms of the agonist according to the invention may include solutions, aqueous or oleaginous suspensions. These suspensions may be formulated according to techniques known in the art using suitable dispersing or wetting agents and suspending agents. The sterile injectable preparation may also be a sterile injectable solution or suspension in a non-toxic pharmaceutically acceptable carrier, such as, e.g., diluent or solvent. Among the acceptable carriers, vehicles and solvents that may be employed, one may cite water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

According to another embodiments, the agonist according to the invention is in an adapted form for a topical administration. Examples of forms adapted for topical administration include, without being limited to, liquid, paste or solid compositions, and more particularly aqueous solutions, drops, dispersions, sprays, microcapsules, nanoparticles, microparticles, polymeric patch, or controlled-release patch, and the like.

In certain embodiments, the agonist according to the invention is to be administered as a monotherapy. As used herein, the term “monotherapy” is intended to mean that the agonist represents the sole therapeutic compound. In said embodiments, the presence of any other therapeutic compound is therefore explicitly excluded.

In some embodiments, said agonist is to be administered with an additional treatment selected in the group consisting of chemotherapy, immunotherapy, radiation, and the like.

As used herein, the term “chemotherapy” refers to a drug treatment that uses chemicals to kill fast-growing cells, in particular cancer cells.

Non-limitative examples of chemotherapy agents include acalabrutinib, alectinib, alemtuzumab, anastrozole, avapritinib, avelumab, belinostat, bevacizumab, bleomycin, blinatumomab, bosutinib, brigatinib, carboplatin, carmustine, cetuximab, chlorambucil, cisplatin copanlisib, cytarabine, daunorubicin, decitabine, dexamethasone, docetaxel, doxorubicin, encorafenib, erdafitinib, etoposide, everolimus, exemestane, fludarabine, 5-fluorouracil, gemcitabine, ifosfamide, imatinib Mesylate, leuprolide, lomustine, mechlorethamine, melphalan, methotrexate, mitomycin, nelarabine, paclitaxel, pamidronate, panobinostat, pralatrexate, prednisolone, ofatumumab, rituximab, temozolomide, topotecan, tositumomab, trastuzumab, vandetanib, vincristine, vorinostat, zanubrutinib, and the like.

As used herein, the term “immunotherapy” refers to a therapy aiming at inducing and/or enhancing an immune response towards a specific target, for example towards infectious agents such as viruses, bacteria, fungi or protozoan parasites, or towards cancer cells.

As used herein, examples of immunotherapies include, without being limited to, vaccination, such as preventive and therapeutic vaccination; adoptive transfer of immune cells, in particular of T cells (such as alpha beta (4) T cells or gamma delta T cells) or NK cells; checkpoint inhibitors; checkpoint agonists; antibodies.

In some embodiments, the immunotherapy is a cancer immunotherapy. As used herein, the term “cancer immunotherapy” refers to an immunotherapy used for the treatment of a cancer, said immunotherapy modulating the immune response of a subject with the aim of inducing and/or stimulating the immune response of the subject towards cancer cells. In some embodiments, the cancer immunotherapy comprises, or consists of, the adoptive transfer of immune cells (ACT), in particular of T cells (such as alpha beta (αβ) T cells or gamma delta (γδ) T cells), NK cells or NK T cells. In some embodiments, the cancer immunotherapy comprises, or consists of, the administration of a checkpoint inhibitor. In some embodiments, the cancer immunotherapy comprises, or consists of, the administration of a checkpoint agonist. In some embodiments, the cancer immunotherapy comprises, or consists of, the administration of an antibody. In some embodiments, the cancer immunotherapy comprises, or consists of, the administration of a therapeutic anti-cancer vaccine.

In certain embodiments, said immunotherapy comprises adoptive transfer of immune cells (ACT), a checkpoint inhibitor, vaccination, the like, and a combination thereof.

As used herein, an adoptive transfer of cells or adoptive cell therapy (or ACT) is defined as the transfer, for example as an infusion, of immune cells to a subject. As a cancer treatment, the adoptive transfer of immune cells to a subject aims at enhancing the subject immune response towards the cancer cells.

In certain embodiments, the transferred immune cells are T cells or natural killer (NK) cells. In some embodiments, the transferred immune cells are T cells, in particular

CD8+T cells, and/or natural killer (NK) cells.

In one embodiment, the transferred immune cells are cytotoxic cells. Examples of cytotoxic cells include natural killer (NK) cells, CD8+T cells, and natural killer (NK) T cells. In one embodiment, the transferred immune cells are natural killer (NK) cells.

In one embodiment, the transferred immune cells are T cells, in particular effector T cells. Examples of effector T cells include CD4+T cells and CD8+T cells. In one embodiment, the transferred immune cells are alpha beta (αβ) T cells. In another embodiment, the transferred immune cells are gamma delta (γδ) T cells. In one embodiment, the transferred immune cells are CD4+T cells, CD8+T cells, or natural killer (NK) T cells, preferably the transferred T cells are CD8+T cells.

In certain embodiments, the transferred immune cells as described hereinabove are antigen-specific immune cells. In one embodiment, the transferred immune cells as described hereinabove are antigen-specific immune cells, wherein said antigen is specifically and/or abundantly expressed by cancer cells. In one embodiment, the transferred immune cells as described hereinabove are tumor-specific immune cells, in other words the transferred immune cells as described hereinabove specifically recognize cancer cells or tumor cells through an antigen specifically and/or abundantly expressed by said cancer cells or tumor cells. In one embodiment, the transferred immune cells as described hereinabove are tumor-specific effector T cells. In one embodiment, the transferred immune cells as described hereinabove are tumor-specific CD8+effector T cells, in particular tumor-specific cytotoxic CD8+T cells. In one embodiment, the transferred immune cells as described hereinabove are tumor-specific cytotoxic cells. In one embodiment, the transferred immune cells as described hereinabove are tumor-specific NK cells.

Examples of tumor-specific antigens, i.e., antigens that are specifically and/or abundantly expressed by cancer cells include, without being limited to, neoantigens (also referred to as new antigens or mutated antigens), 9D7, ART4, β-catenin, BING-4, Bcr-abl, BRCA1/2, calcium-activated chloride channel 2, CDK4, CEA (carcinoembryonic antigen), CML66, Cyclin B1, CypB, EBV (Epstein-Barr virus) associated antigens (such as LMP-1, LMP-2, EBNA1 and BARF1), EG1-RvIII, Ep-CAM, EphA3, fibronectin, Gp100/pme117, Her2/neu, HPV (human papillomavirus) E6, HPV E7, hTERT, IDH1, IDH2, immature laminin receptor, MC1R, Melan-A/MART-1, MART-2, mesothelin, MUC1, MUC2, MUM-1, MUM-2, MUM-3, NY-ESO-1/LAGE-2, p53, PRAME, prostate-specific antigen (PSA), PSMA (prostate-specific membrane antigen), Ras, SAP-1, SART-I, SART-2, SART-3, SSX-2, survivin, TAG-72, telomerase, TRP-1/-2, tyrosinase, WT1, antigens of the BAGE family, antigens of the CAGE family, antigens of the GAGE family, antigens of the MAGE family, antigens of the SAGE family, and antigens of the XAGE family

As used herein, neoantigens (also referred to as new antigens or mutated antigens) correspond to antigens derived from proteins that are affected by somatic mutations or gene rearrangements acquired by the tumors. Neoantigens may be specific to each individual subject and thus provide targets for developing personalized immunotherapies. Examples of neoantigens include for example, without being limited to, the R24C mutant of CDK4, the R24L mutant of CDK4, KRAS mutated at codon 12, mutated p53, the V600E mutant of BRAF and the R132H mutant of IDH1.

In one embodiment, the transferred immune cells as described hereinabove are specific for a tumor antigen selected from the group comprising or consisting of the class of CTAs (cancer/testis antigens, also known as MAGE-type antigens), the class of neoantigens and the class of viral antigens.

As used herein, the class of CTAs corresponds to antigens encoded by genes that are expressed in tumor cells but not in normal tissues except in male germline cells. Examples of CTAs include, without being limited to, MAGE-A1, MAGE-A3, MAGE-A4, MAGE-C2, NY-ESO-1, PRAME and SSX-2.

As used herein, the class of viral antigens corresponds to antigens derived from viral oncogenic proteins. Examples of viral antigens include, without being limited to, HPV (human papillomavirus) associated antigens such as E6 and E7, and EBV (Epstein-Barr virus) associated antigens such as LMP-1, LMP-2, EBNA1 and BARF1.

In one embodiment, the transferred immune cells as described hereinabove are autologous immune cells, in particular autologous T cells. In another embodiment, the transferred immune cells as described hereinabove are allogenic (or allogenous) immune cells, in particular allogenic NK cells.

For example, autologous T cells can be generated ex vivo either by expansion of antigen-specific T cells isolated from the subject or by redirection of T cells of the subject through genetic engineering.

In one embodiment, the immune cells to be infused are modified ex vivo, in particular with RNA interference (also known as RNAi), before being infused to the subject.

Methods to isolate T cells from a subject, in particular antigen-specific T cells, e.g., tumor-specific T cells, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Methods to expand T cells ex vivo are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71). Protocols for infusion of T cells in a subject, including pre-infusion conditioning regimens, are well-known in the art (see for example Rosenberg & Restifo, 2015, Science 348, 62-68; Prickett et al., 2016, Cancer Immunol Res 4, 669-678; or Hinrichs & Rosenberg, 2014, Immunol Rev 257, 56-71).

In some embodiments, said immune cells are selected in the group comprising T cells, in particular CD8+T cells and CAR T cells; natural killer (NK) cells, in particular CAR NK cells; the like; and a combination thereof.

As used herein, CAR immune cell therapy is an adoptive cell therapy wherein the transferred cells are immune cells as described hereinabove, such as T cells or NK cells, genetically engineered to express a chimeric antigen receptor (CAR). As a cancer treatment, the adoptive transfer of CAR immune cells to a subject aims at enhancing the subject immune response towards the cancer cells.

CARs are synthetic receptors consisting of a targeting moiety that is associated with one or more signaling domains in a single fusion molecule or in several molecules. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are usually derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Thus, signaling domains from co-stimulatory molecules including CD28, OX-40 (CD134), and 4-1BB (CD137) have been added alone (second generation) or in combination (third generation) to enhance survival and increase proliferation of CAR modified T cells.

Thus, in one embodiment, the transferred T cells as described hereinabove are CAR T cells. The expression of a CAR allows the T cells to be redirected against a selected antigen, such as an antigen expressed at the surface of cancer cells. In one embodiment, the transferred CAR T cells recognize a tumor-specific antigen.

In another embodiment, the transferred NK cells as described hereinabove are CAR NK cells. The expression of a CAR allows the NK cells to be redirected against a selected antigen, such as an antigen expressed at the surface of cancer cells. In one embodiment, the transferred CAR NK cells recognize a tumor-specific antigen.

Examples of tumor-specific antigens are mentioned hereinabove.

In one embodiment, the transferred CAR T cells or CAR NK cells recognize a tumor-specific antigen selected from the group comprising or consisting of EGFR and in particular EGFRvIII, mesothelin, PSMA, PSA, CD47, CD70, CD133, CD171, CEA, FAP, GD2, HER2, IL-13Rα, αvβ6 integrin, ROR1, MUC1, GPC3, EphA2, CD19, CD21, and CD20.

In one embodiment, the CAR immune cells as described hereinabove are autologous CAR immune cells, in particular autologous CAR T cells. In another embodiment, the CAR immune cells as described hereinabove are allogenic (or allogenous) CAR immune cells, in particular allogenic CAR NK cells.

As used herein, a checkpoint inhibitor therapy is defined as the administration of at least one checkpoint inhibitor to the subject.

Checkpoint inhibitors (CPI, that may also be referred to as immune checkpoint inhibitors or ICI) block the interactions between inhibitory receptors expressed on T cells and their ligands. As a cancer treatment, checkpoint inhibitor therapy aims at preventing the activation of inhibitory receptors expressed on T cells by ligands expressed by the tumor cells. Checkpoint inhibitor therapy thus aims at preventing the inhibition of T cells present in the tumor, i.e., tumor infiltrating T cells, and thus at enhancing the subject immune response towards the tumor cells.

Examples of checkpoint inhibitors include, without being limited to, inhibitors of the cell surface receptor PD-1 (programmed cell death protein 1), also known as CD279 (cluster differentiation 279); inhibitors of the ligand PD-L1 (programmed death-ligand 1), also known as CD274 (cluster of differentiation 274) or B7-H1 (B7 homolog 1); inhibitors of the cell surface receptor CTLA4 or CTLA-4 (cytotoxic T-lymphocyte-associated protein 4), also known as CD152 (cluster of differentiation 152); inhibitors of IDO (indoleamine 2,3-dioxygenase) and inhibitors of TDO (tryptophan 2,3-dioxygenase); inhibitors of LAG-3 (lymphocyte-activation gene 3), also known as CD223 (cluster differentiation 223); inhibitors of TIM-3 (T-cell immunoglobulin and mucin-domain containing-3), also known as HAVCR2 (hepatitis A virus cellular receptor 2) or CD366 (cluster differentiation 366); inhibitors of TIGIT (T cell immunoreceptor with Ig and ITIM domains), also known as VSIG9 (V-Set And Immunoglobulin Domain-Containing Protein 9) or VSTM3 (V-Set And Transmembrane Domain-Containing Protein 3); inhibitors of BTLA (B and T lymphocyte attenuator), also known as CD272 (cluster differentiation 272); inhibitors of CEACAM-1 (carcinoembryonic antigen-related cell adhesion molecule 1) also known as CD66a (cluster differentiation 66a).

In one embodiment, the at least one checkpoint inhibitor is selected from the group comprising or consisting of inhibitors or PD-1, inhibitors of PD-L1, inhibitors of CTLA-4 and any mixtures thereof.

In one embodiment, the at least one checkpoint inhibitor is selected from the group comprising or consisting of pembrolizumab, nivolumab, cemiplimab, tislelizumab, spartalizumab, ABBV-181, JNJ-63723283, BI 754091, MAG012, TSR-042, AGEN2034, avelumab, atezolizumab, durvalumab, LY3300054, ipilimumab, tremelimumab, and any mixtures thereof.

In one embodiment, the at least one checkpoint inhibitor is selected from the group comprising or consisting of pembrolizumab, nivolumab, cemiplimab, tislelizumab, spartalizumab, ABBV-181, JNJ-63723283, avelumab, atezolizumab, durvalumab, ipilimumab, tremelimumab, and any mixtures thereof.

In one embodiment, the at least one checkpoint inhibitor is an inhibitor of PD-1, also referred to as an anti-PD-1. Inhibitors of PD-1 may include antibodies targeting PD-1, in particular monoclonal antibodies, and non-antibody inhibitors such as small molecule inhibitors. Examples of inhibitors of PD-1 include, without being limited to, pembrolizumab, nivolumab, cemiplimab, tislelizumab, spartalizumab, ABBV-181, JNJ-63723283, BI 754091, MAG012, TSR-042, and AGEN2034. Pembrolizumab is also known as MK-3475, MK03475, lambrolizumab, or SCH-900475. The trade name of pembrolizumab is Keytruda®. Nivolumab is also known as ONO-4538, BMS-936558, MDX1106, or GTPL7335. The trade name of nivolumab is Opdivo®. Cemiplimab is also known as REGN2810 or REGN-2810. Tislelizumab is also known as BGB-A317. Spartalizumab is also known as PDR001 or PDR-001. In one embodiment, the at least one checkpoint inhibitor is selected from the group comprising or consisting of pembrolizumab, nivolumab, cemiplimab, tislelizumab, spartalizumab, ABBV-181, JNJ-63723283, BI 754091, MAG012, TSR-042, AGEN2034, and any mixtures thereof. In one embodiment, the at least one checkpoint inhibitor is selected from the group comprising or consisting of pembrolizumab, nivolumab, cemiplimab, tislelizumab, spartalizumab, ABBV-181, JNJ-63723283, and any mixtures thereof.

In one embodiment, the at least one checkpoint inhibitor is an inhibitor of PD-L1, also referred to as an anti-PD-L1. Inhibitors of PD-L1 may include antibodies targeting PD-L1, in particular monoclonal antibodies, and non-antibody inhibitors such as small molecule inhibitors. Examples of inhibitors of PD-L1 include, without being limited to, avelumab, atezolizumab, durvalumab and LY3300054. Avelumab is also known as MSB0010718C, MSB-0010718C, MSB0010682, or MSB-0010682. The trade name of avelumab is Bavencio®. Atezolizumab is also known as MPDL3280A (clone YW243.55.S70), MPDL-3280A, RG-7446 or RG7446. The trade name of atezolizumab is Tecentriq®. Durvalumab is also known as MEDI4736 or MEDI-4736. The trade name of durvalumab is Imfinzi® In one embodiment, the at least one checkpoint inhibitor is selected from the group comprising or consisting of avelumab, atezolizumab, durvalumab, LY3300054, and any mixtures thereof. In one embodiment, the at least one checkpoint inhibitor is selected from the group comprising or consisting of avelumab, atezolizumab, durvalumab, and any mixtures thereof.

In one embodiment, the at least one checkpoint inhibitor is an inhibitor of CTLA-4, also referred to as an anti-CTLA-4. Inhibitors of CTLA-4 may include antibodies targeting CTLA-4, in particular monoclonal antibodies, and non-antibody inhibitors such as small molecule inhibitors. Examples of inhibitors of CTLA-4 include, without being limited to, ipilimumab and tremelimumab. Ipilimumab is also known as BMS-734016, MDX-010, or MDX-101. The trade name of ipilimumab is Yervoy®. Tremelimumab is also known as ticilimumab, CP-675, or CP-675,206. In one embodiment, the at least one checkpoint inhibitor is selected from the group comprising or consisting of ipilimumab, tremelimumab, and any mixtures thereof.

In one embodiment, the at least one checkpoint inhibitor is an inhibitor of IDO or an inhibitor of TDO, also referred to as an anti-IDO or anti-TDO, respectively. Examples of inhibitors of IDO include, without being limited to, 1-methyl-D-tryptophan (also known as indoximod), epacadostat (also known as INCB24360), navoximod (also known as IDO-IN-7 or GDC-0919), linrodostat (also known as BMS-986205), PF-06840003 (also known as EOS200271), TPST-8844, and LY3381916.

As used herein, an antibody therapy is defined as the administration of at least one antibody to the subject.

As a cancer treatment, antibody therapy aims at enhancing the subject immune response towards the cancer cells, notably by targeting cancer cells for destruction, by stimulating the activation of T cells present in the tumor or by preventing the inhibition of T cells present in the tumor, or at inhibiting the growth or spreading of cancer cells.

As used herein, “antibody therapy” may include the administration of monoclonal antibodies, polyclonal antibodies, multiple-chain antibodies, single-chain antibodies, single-domain antibodies, antibody fragments, antibody domains, antibody mimetics or multi-specific antibodies such as bispecific antibodies.

In one embodiment, the antibody is for or aims at targeting cancer cells or tumor cells for destruction.

Examples of antibodies, in particular monoclonal antibodies, targeting cancer cells or tumor cells for destruction include tumor-specific antibodies, in particular tumor-specific monoclonal antibodies. Examples of tumor-specific antibodies, include, without being limited to, antibodies targeting cell surface markers of cancer cells or tumor cells, antibodies targeting proteins involved in the growth or spreading of cancer cells or tumor cells.

In one embodiment, the antibody is for or aims at stimulating the activation of T cells present in the tumor.

Examples of antibodies, in particular monoclonal antibodies, stimulating the activation of T cells present in the tumor include, without being limited to, anti-CD137 antibodies and anti-OX40 antibodies as described hereinabove.

In one embodiment, the antibody is for or aims at preventing the inhibition of T cells present in the tumor.

Examples of antibodies, in particular monoclonal antibodies, preventing the inhibition of T cells present in the tumor include, without being limited to, anti-PD-1 antibodies (such as pembrolizumab, nivolumab, cemiplimab, tislelizumab, and spartalizumab), anti-PD-L1 antibodies (such as avelumab, atezolizumab, and durvalumab) and anti-CTLA-4 antibodies (such as ipilimumab and tremelimumab) as described hereinabove.

In one embodiment, the antibody is for or aims at inhibiting the growth or spreading of cancer cells.

Examples of antibodies inhibiting the growth or spreading of cancer cells include, without being limited to, anti-HER2 antibodies (such as trastuzumab).

As used herein, the term “vaccination” refers to the use of a preparation comprising a substance or a group of substances (i.e., a vaccine) meant to induce and/or enhance in a subject a targeted immune response towards cancer cells. Prophylactic vaccination is used to prevent a subject from ever having a particular disease or to only have a mild case of the disease. Therapeutic vaccination is intended to treat a particular disease in a subject, for example cancers. For example, therapeutic anti-cancer vaccines may comprise a tumor-associated antigen or tumor-associated antigens, aiming at inducing and/or enhancing a cell-mediated immune response, in particular a T cell immune response, directed towards the cancer cells expressing said tumor-associated antigen(s).

As used herein, a “therapeutic vaccine” is defined as the administration of at least one tumor-specific antigen (e.g., synthetic long peptides or SLP), or of the nucleic acid encoding said tumor-specific antigen; the administration of recombinant viral vectors selectively entering and/or replicating in tumor cells; the administration of tumor cells;

and/or the administration of immune cells (e.g., dendritic cells) engineered to present tumor-specific antigens and trigger an immune response against these antigens.

As a cancer treatment, therapeutic vaccines aim at enhancing the subject immune response towards the tumor cells.

Examples of therapeutic vaccines aiming at enhancing the subject immune response towards the tumor cells include, without being limited to, viral-vector based therapeutic vaccines such as adenoviruses (e.g., oncolytic adenoviruses), vaccinia viruses (e.g., modified vaccinia Ankara (MVA)), alpha viruses (e.g., Semliki Forrest Virus (SFV)), measles virus, Herpes simplex virus (HSV), and coxsackievirus; synthetic long peptide (SLP) vaccines; RNA-based vaccines, and dendritic cell vaccines.

In certain embodiments, said chemotherapy and/or immunotherapy is to be administered separately or concomitantly with said agonist.

In some embodiments, the additional treatment comprises the administration of an agent that increases the expression of alpha-2 adrenergic receptors, preferably at the RNA level. As used herein, the expression “increases the expression of alpha-2 adrenergic receptors” is intended to mean that the agent promotes the synthesis of alpha-2 adrenergic receptors, which results in an increased number of alpha-2 adrenergic receptors in the presence of said agent. Illustratively, the increase may represent at least about 10% increase of the number of alpha-2 adrenergic receptors.

Within the scope of the invention, the expression “about 10%” encompasses about 10%, 20%, 30%, 40%, 50%; 60%, 70%, 80%, 90%, 100%, 150%, 200%, 250%, 300%, 350%, 400%, 450%, 500%, 550%, 600%, 650%, 700%, 750%, 800%, 850%, 900%, 950%, about 1,000%, or more. In practice, the increase in the number of alpha-2 adrenergic receptors may be assessed by any suitable methods, or a method adapted therefrom, in particular immunostaining, western blotting, and the like.

In certain embodiments, the agent that increases the expression of alpha-2 adrenergic receptors may be an activating RNA.

In certain embodiments, said cancer is selected in the group consisting of myelofibrosis, acute lymphoblastic leukemia, acute myeloblastic leukemia adrenal gland carcinoma, bile duct cancer, bladder cancer, breast cancer, cervical cancer, colorectal cancer, endometrial cancer, esophageal cancer, gastric cancer, gastrointestinal stromal tumors, glioblastoma, head and neck cancer, hepatocellular carcinoma, Hodgkin's lymphoma, kidney cancer, lung cancer, melanoma, Merkel cell skin cancer, mesothelioma, multiple myeloma, myeloproliferative disorders, non-Hodgkin lymphoma, ovarian cancer, pancreatic cancer, prostate cancer, salivary gland cancer, sarcoma, squamous cell carcinoma, testicular cancer, thyroid cancer, urothelial carcinoma, and uveal melanoma.

In some embodiments, said cancer is a blood cancer or a solid cancer.

As used herein, the term “blood cancer”, also referred to as “hematologic cancer”, encompasses any cancer involving uncontrolled proliferation of blood cells, in particular white blood cells. Blood cancers includes leukemia, lymphoma (Hodgkin and non-Hodgkin lymphomas) and myeloma.

In certain embodiments, the cancer is a blood cancer. In some embodiments, said cancer is a blood cancer selected in the group consisting of myelofibrosis, Hodgkin's disease, immunoblastic lymphadenopathy, lymphoma, chronic lymphocytic leukemia, acute leukemia, and the like. In certain embodiments, the blood cancer is a T cell lymphoma or a myeloma. In some embodiments, the blood cancer is myelofibrosis.

As used herein, the term “solid cancer” encompasses any cancer (also referred to as malignancy) that forms a discrete tumor mass, as opposed to cancers (or malignancies) that diffusely infiltrate a tissue without forming a mass.

In certain embodiments, the cancer is a solid cancer. In one embodiment, the solid cancer is a carcinoma.

In some embodiments, the solid cancer is selected in the group consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, fibrosarcoma, glioblastoma, prostate carcinoma, ovarian cancer and pancreatic carcinoma.

In certain embodiments, the solid cancer is not a cholangiocarcinoma.

In some embodiments, the solid cancer is selected in the group consisting of colon carcinoma, ovarian cancer, melanoma, breast carcinoma, liver carcinoma, lung carcinoma, renal carcinoma, prostate carcinoma and fibrosarcoma.

In one embodiment, the solid cancer is selected in the group consisting of colon carcinoma, ovarian cancer, prostate carcinoma and melanoma.

In certain embodiments, the agonist is for use for reducing the volume and/or the weight of a solid tumor.

Another aspect of the invention pertains to a pharmaceutical composition comprising an alpha-2 adrenergic receptor agonist, as defined herein, and a pharmaceutically acceptable carrier, for use in the prevention and/or the treatment of cancer.

In a further aspect, the invention pertains to a pharmaceutical composition comprising an alpha-2 adrenergic receptor agonist, as defined herein, and a pharmaceutically acceptable carrier, for use in the treatment of cancer.

Pharmaceutically acceptable carriers that may be used in the pharmaceutical composition of the invention include, without being not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol, wool fat, the like, and combinations thereof.

In some aspect, the invention also relates to the use of an alpha-2 adrenergic receptor agonist for the preparation and/or the manufacture of a medicament for the prevention and/or the treatment of cancer. The invention further pertains to the use of an alpha-2 adrenergic receptor agonist for the preparation and/or the manufacture of a medicament for the treatment of cancer.

In another aspect, the invention relates to a method for the prevention and/or the treatment of cancer in an individual in need thereof comprising the administration of a therapeutic effective amount of an alpha-2 adrenergic receptor agonist, as an active agent. The invention also relates to a method for the treatment of cancer in an individual in need thereof comprising the administration of a therapeutic effective amount of an alpha-2 adrenergic receptor agonist, as an active agent.

A still other aspect of the invention relates to a method for reducing the volume and/or the weight of a solid tumor in an individual in need thereof comprising the administration of a therapeutic effective amount of an alpha-2 adrenergic receptor agonist, as an active agent.

In practice, the volume and/or the weight of a solid tumor may be evaluated by the mean of any suitable method known in the state of the art, or any method adapted therefrom. Illustratively, the volume and/or the weight of the solid tumor may be evaluated by echography, radiography, MRI, Pet-Scan, and the like.

In some embodiments, the volume of the solid tumor may be expressed in mm³, and estimated following the formula: Volume=width²×length/2. In certain embodiments, the weight of the solid tumor in expressed in g.

In some embodiments, effective reduction of the volume and/or the weight of a solid tumor may be achieved when said reduction reaches at least about 1.2-fold, as compared to the volume and/or the weight of said untreated solid tumor.

Within the scope of the instant invention, the term “about 1.2-fold” includes 1.2, 1.3, 1.4, 1.5, 1.6, 1.7, 1.8, 1.9, 2.0, 2.2, 2.4, 2.6, 2.8, 3.0, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7.0, 7.2, 7.4, 7.6, 7.8, 8.0, 8.2, 8.4, 8.6, 8.8, 9.0, 9.2, 9.4, 9.6, 9.8, 10, 12, 14, 16, 18, 20, 25, 30, 35, 40, 45, 50, 55, 60, 65, 70, 75, 80, 85, 90, 95, 100, 120, 140, 160, 180, 200, 220, 240, 260, 280, 300, 320, 340, 360, 380, 400, 420, 440, 460, 480, 500, 550, 600, 650, 700, 750, 800, 850, 900, 950, 1,000-fold, or more.

In certain embodiments, the alpha-2 adrenergic receptor agonist is the sole active agent for the prevention and/or the treatment of cancer.

In one aspect, the invention pertains to a kit for preventing and/or treating cancer comprising:
an alpha-2 adrenergic receptor agonist or a pharmaceutical composition, as defined herein, and
a means to administer the alpha-2 adrenergic receptor agonist or the pharmaceutical composition.

One aspect of the invention relates to a kit for treating cancer comprising:
an alpha-2 adrenergic receptor agonist or a pharmaceutical composition, as defined herein, and
a means to administer the alpha-2 adrenergic receptor agonist or the pharmaceutical composition.

In a further aspect, the invention concerns a kit for treating and/or preventing cancer comprising:
an alpha-2 adrenergic receptor agonist or a pharmaceutical composition, as defined herein, and
an anti-tumor compound, in particular selected in the group consisting of a chemotherapy agent and an immunotherapy agent.

A further aspect of the invention concerns a kit for treating cancer comprising:
an alpha-2 adrenergic receptor agonist or a pharmaceutical composition, as defined herein, and
an anti-tumor compound, in particular selected in the group consisting of a chemotherapy agent and an immunotherapy agent.

### BRIEF DESCRIPTION OF THE DRAWINGS

FIG. 1 is set of curves showing that clonidine promotes a decrease of the tumor volume in a model of colon carcinoma. BALB/c mice bearing CT26 colon carcinomas received daily injections of vehicle control (black circles), clonidine (5 mg/kg, i.p.) (inverted grey triangles), clonidine (5 mg/kg, i.p.) plus phentolamine (5 mg/kg, i.p.) (black squares), or clonidine (5 mg/kg, i.p.) plus propranolol (5 mg/kg, i.p.) (grey triangles), when the tumor size was around 50 mm³, until the day of sacrifice. Tumor volume is expressed in mm³. *** p<0.001.

FIG. 2 is set of curves showing that guanabenz promotes a decrease of the tumor volume in a model of colon carcinoma. BALB/c mice bearing CT26 colon carcinomas received daily injections of vehicle control (black circles), guanabenz (5 mg/kg, i.p.) (black squares), or guanabenz (5 mg/kg, i.p.) plus phentolamine (5 mg/kg, i.p.) (grey circles), when the tumor size was around 50 mm³, until the day of sacrifice. Tumor volume is expressed in mm³. * p<0.05.

FIG. 3 is set of curves showing that the effect of clonidine in promoting a decrease of the tumor volume in a model of colon carcinoma is strongly attenuated by yohimbine. BALB/c mice bearing CT26 colon carcinomas received daily injections of vehicle control (black circles), clonidine (5 mg/kg, i.p.) (black squares), clonidine (5 mg/kg, i.p.) plus yohimbine (10 mg/kg, i.p.) (grey triangles), or yohimbine (10 mg/kg, i.p.) (black inverted triangles), when the tumor size was around 50 mm³, until the day of sacrifice. Tumor volume is expressed in mm³. ** p<0.01.

FIG. 4 is set of curves showing that the effect of guanabenz in promoting a decrease of the tumor volume in a model of colon carcinoma is strongly attenuated by yohimbine. BALB/c mice bearing CT26 colon carcinomas received daily injections of vehicle control (black circles), guanabenz (5 mg/kg, i.p.) (black diamonds), guanabenz (5 mg/kg, i.p.) plus yohimbine (10 mg/kg, i.p.) (grey circles), or yohimbine (10 mg/kg, i.p.) (black inverted triangles), when the tumor size was around 50 mm³, until the day of sacrifice. Tumor volume is expressed in mm³. *** p<0.001.

FIGS. 5A-B is a set of histograms showing the increase of tumor infiltration of CD8+T cells upon administration of romifidine or clonidine. TiRP-tumor bearing mice received adoptive cell transfer (ACT) of 10 million of P1A-specific activated CD8+T cells. Daily injections of romifidine (5 mg/kg i.p., FIG. 5A) or clonidine (5 mg/kg i.p., FIG. 5B) were administered from the day of the ACT, when the tumor size was around 500 mm³, until the day of sacrifice. Tumor infiltration of CD8+T cells is expressed as the %CD8+T cells among total CD45+T cells. * p<0.05.

FIGS. 6A-B is a set of curves and histograms showing the tumor growth (FIG. 6A) in TiRP+/+ mice that have received daily injections of clonidine (5 mg/kg, i.p., curve 2) or vehicle (PBS, i.p., curve 1), after ACT with 10 million P1A-specific activated CD8+T cells, which was performed when the tumor size was around 400 mm³. Tumor infiltration of CD8+cells evaluated at the day of sacrifice by FACS (FIG. 6B).

FIGS. 7A-B is a set of curves and histogram showing that clonidine strongly increased the anti-tumor efficacy of human PBMC against ovarian carcinoma cells SKOV3. SKOV3 human ovarian tumor cells were injected subcutaneously in immune deficient NSG (NOD Scid-gamma) mice. When tumors were palpable (50 mm³), tumor-bearing mice received adoptive cell transfer of 3 million of allogeneic human PBMC (i.v.) and were injected daily with clonidine (5 mg/kg, i.p.; grey squares; curve 2) or vehicle (PBS, i.p.; black circles; curve 1) until sacrifice. (FIG. 7A) depicts the evolution of the tumor volume (in mm³) during the time course upon treatment (in days). (FIG. 7B) depicts the comparison of the clonidine treatment combined with ACT (grey bar) as compared to ACT only (black bar), with respect to the tumor weight (in g). * p<0.05; **** p<0.0001.

FIGS. 8A-C is a set of histograms showing that clonidine cooperates with the therapeutic efficacy of human T cells in a human xenograft model. SKOV3 human ovarian tumor cells were injected subcutaneously in immune deficient NSG mice. When tumors were palpable, tumor-bearing mice received adoptive cell transfer of 3 million of allogeneic human PBMC (i.v.) and were injected daily with clonidine (5 mg/kg, i.p.; grey squares) or vehicle (PBS, i.p.; black circles) until sacrifice. Following clonidine administration (5 mg/kg, i.p.), infiltration of both CD8+(FIG. 8A) and CD4+(FIG. 8C) human T cells into human SKOV3 tumors was monitored, as well as the expression of CD44 on tumor-infiltrated CD8+T cells (FIG. 8B). * p<0.05.

FIG. 9 is a set of curves showing that clonidine strongly sensitized immune-resistant autochthonous melanoma tumors (TiRP) to adoptive cell transfer (ACT). TiRP-tumor bearing mice received adoptive cell transfer (ACT) of 10 million P1A-specific activated CD8+T cells and daily injections of vehicle control (black circles), clonidine (5 mg/kg, i.p.) (black squares), or clonidine (5 mg/kg, i.p.) plus phentolamine (5 mg/kg, i.p.) (grey triangles), when the tumor size was around 500 mm³, until the day of sacrifice. Tumor volume is expressed in mm³. ** p<0.01.

FIGS. 10A-B is a set of curves showing the tumor growth of B16F1 melanoma in C57BL/6J (FIG. 10A) or immunodeficient NSG mice (FIG. 10B) treated with PBS (Control, curve 1) or clonidine (5 mg/kg, i.p., curve 2).

FIGS. 11A-B is a set of curves showing the tumor growth of B16F1 melanoma in C57BL/6J (FIG. 11A) or NSG mice (FIG. 11B) treated with PBS (Control, curve 1) or guanfacine (5 mg/kg, i.p., curve 2).

FIGS. 12A-B is a set of curves and histograms showing the tumor growth (FIG. 12A) in TiRP+/+ mice that have received daily injection of guanfacine (5 mg/kg, i.p., curve 2) or vehicle (PBS, i.p., curve 1), after ACT with 10 million P1A-specific activated CD8+T cells, which was performed when the tumor size was around 400 mm³. Tumor infiltration of CD8+cells evaluated at the day of sacrifice by FACS (FIG. 12B).

FIGS. 13A-E is a set of curves and histograms showing that clonidine or guanabenz treatment benefits myelofibrosis (blood cancer) induced by JAK2V617F mutant bone marrow transplantation. FIGS. 13A-C: Trends of JAK2 V617F mutant allele burden variation (as reflected by CD45.2 donor chimerism in the blood of primary recipients) in mice treated with PBS (panel A), clonidine (5 mg/kg i.p.; panel B) or guanabenz (5 mg/kg i.p.; panel C) over 7 days. FIG. 13D is a histogram showing that clonidine or guanabenz treatment significantly reduces the platelet (PLT) counts (m/mm³) in primary myelofibrosis, induced by JAK2V617F mutant bone marrow transplantation. ** P<0.01. FIG. 13E is a histogram showing that clonidine treatment significantly reduces the platelet (PLT) counts (m/mm³) in myelofibrosis induced by romiplostim. * P<0.05; ns: not significant.

FIGS. 14A-B is a set of curves showing the tumor growth (melanoma) in TiRP+/+ mice that have received daily injections of guanfacine (5 mg/kg, i.p. triangles; FIG. 14A), clonidine (5 mg/kg, i.p., circles; FIG. 14B), or control (PBS, i.p., squares), when the tumor size was around 250 mm³ until sacrifice.

FIGS. 15A-C is a set of curves and histograms showing the tumor growth (FIG. 15A) of CT26 colon cancer in C57BL/6J mice treated with PBS (Control, circles), clonidine (squares), clonidine mixed with yohimbine (dark grey inverted triangles), or yohimbine alone (light grey inverted triangles). Tumor infiltration of CD8+cells evaluated at the day of sacrifice by FACS (FIG. 15B). Tumor growth of CT26 colon cancer in NSG mice treated with PBS (Control), clonidine, clonidine mixed with yohimbine, or yohimbine alone (FIG. 15C).

FIGS. 16A-C is a set of curves and histograms showing the tumor growth of CT26 colon cancer in C57BL/6J mice treated with PBS (Control, circles), guanabenz (diamonds), guanabenz mixed with yohimbine (light grey inverted triangles), or yohimbine alone (dark grey inverted triangles) (FIG. 16A); the tumor infiltration of CD8+ cells evaluated at the day of sacrifice by FACS (FIG. 16B); and the tumor growth of CT26 colon cancer in NSG mice treated with PBS (Control), guanabenz, guanabenz mixed with yohimbine, or yohimbine alone (FIG. 16C).

FIGS. 17A-B is a set of curves showing the tumor growth of LS411N ovarian cancer in mice treated with PBS (Control, curve 1), clonidine (curve 2), clonidine mixed with yohimbine (curve 3), or yohimbine alone (curve 4) (FIG. 17A); with PBS (Control), guanabenz, guanabenz mixed with yohimbine, or yohimbine alone (FIG. 17B); upon adoptive transfer of allogeneic human PBMC. Adoptive transfer of human PBMC was performed when tumor size reaches 100 mm³.

FIGS. 18A-B is a set of curves showing the tumor growth of MC38 colon cancer in Wild type (FIG. 18A) or Adra2a-KO mice (FIG. 18B) treated with PBS (Control; curves 1) or clonidine (curves 2).

FIGS. 19A-B is a set of curves showing the tumor growth of MC38 colon cancer in Wild type (FIG. 19A) or Adra2a-KO mice (FIG. 19B) treated with PBS (Control; curves 1) or guanabenz (curves 2).

FIGS. 20A-B is a set of curves showing the tumor growth of B16F10-OVA melanoma in Wild type (FIG. 20A) or Adra2a-KO mice (FIG. 20B) treated with PBS (Control; curves 1) or clonidine (curves 2).

FIGS. 21A-B is a set of curves showing the tumor growth of B16F10-OVA melanoma in Wild type (FIG. 21A) or Adra2a-KO mice (FIG. 21B) treated with PBS (Control; curves 1) or guanabenz (GBZ, curves 2).

FIGS. 22A-H is a set of curves showing the tumor growth of mice treated with PBS (Control, circles) or clonidine (squares) in different tumor models: such as mammary gland tumor (FIG. 22A), hepatocellular carcinoma (FIG. 22B), T cell lymphoma (FIG. 22C), lung cancer (FIG. 22D), myeloma (FIG. 22E), mammary adenocarcinoma (FIG. 22F), renal cancer (FIG. 22G) and fibrosarcoma (FIG. 22H).

FIGS. 23A-H is a set of curves showing the tumor growth of mice treated with PBS (Control, circles) or guanabenz (GBZ, triangles) in different tumor models: such as mammary gland tumor (FIG. 23A), hepatocellular carcinoma (FIG. 23B), T cell lymphoma (FIG. 23C), lung cancer (FIG. 23D), myeloma (FIG. 23E), mammary adenocarcinoma (FIG. 23F), renal cancer (FIG. 23G) and fibrosarcoma (FIG. 23H).

FIGS. 24A-B is a set of curves showing the tumor growth of NSG mice that received allogeneic human PBMC and a prostate cancer as xenograft, and further treated with PBS (Control, circles) or clonidine (squares) (FIG. 24A) or with PBS (Control, circles) or guanabenz (GBZ, triangles) (FIG. 24B)

FIGS. 25A-B is a set of curves showing the tumor growth of MC38 (FIG. 25A) or CT26 (FIG. 25B) colon cancer in mice treated with PBS (Control; closed circles), clonidine (5 mg/kg, i.p; closed squares) or apraclonidine (5 mg/kg, i.p; closed triangles).

### EXAMPLES

The present invention is further illustrated by the following examples.

### Example 1

### Materials and Methods

1—Mice

TiRP mice have been created by crossing Ink4a/Are^{flox/flox} mice with mice carrying a transgenic construct controlled by the tyrosinase promoter and driving the expression of H-Ras ^{12V} and Trapl a which encodes a MAGE-type tumor antigen P1A; the promoter is separated from the coding region by a stop cassette made of a floxed self-deleting CreER (Huijbers et al., 2006, Cancer Res 66, 3278-3286). Those mice were backcrossed to a B10.D2 background and bred to homozygosity. TCRP1A mice heterozygous for the H-2Ld/P1A35-43-specific TCR transgene were kept on the B10.D2;Rag1^{-/-} background (Shanker et al., 2004, J Immunol 172, 5069-5077). BALB/c mice were used in tumor transplantation experiments. TiRP-derived T429.11 transplanted melanoma model: T429.11 clone was derived from an induced Amela TiRP tumor referred to as T429. It was cloned from the T429 induced melanoma primary tumor line. Two million of T429.11 tumor cells were injected subcutaneously into recipient mice for tumor establishment (Zhu et al., 2017, Nat Commun. 10; 8(1):1404). Adra2a tm1Bkk mice (Strain Name: B6.129-Adra2a tm1Bkk /J, # 004367) were obtained from Jackson Laboratory (Bar Harbor, Me., USA).

All mice used in this study were produced under specific pathogen free (SPF) conditions at the animal facility of the de Duve Institute. All the rules concerning animal welfare have been respected according to the 2010/63/EU Directive. All procedures were performed with the approval of the local Animal Ethical Committee, with reference 2015/UCL/MD/15.

2—Tumor Induction with 40H-Tamoxifen

A fresh solution of 40H-Tamoxifen was prepared by dissolving 40H-Tamoxifen (Imaginechem®) in 100% ethanol and mineral oil (ratio 1:9) followed by 30 min sonication, and injected subcutaneously (2 mg/200 uL per mouse) in the neck area of gender-matched 7 weeks old TiRP mice. Tumor appearance was monitored daily and tumors were measured three times/week. Tumor volume (in mm³) was calculated by the following formula: Volume=width²×length/2.

3—Cell Cultures

SKOV3 human ovarian cancer cells and CT26 murine colon carcinoma cells were cultured in IMDM medium supplemented with 10% Fetal bovine serum (FBS).

4—Drug Administration

Mice received a daily intra-peritoneal injection of guanabenz (5 mg/kg) or vehicle (PBS) from the day of randomization (and the day of ACT when applicable) until the day of sacrifice. For the co-administration of adrenergic receptor agonist and antagonist, BALB/c mice bearing CT26 colon carcinomas received daily injections of vehicle control, guanabenz (5 mg/kg, i.p.), or guanabenz (5 mg/kg, i.p.) plus yohimbine (10 mg/kg, i.p.), when the tumor size was around 50 mm³, until the day of sacrifice.

5—Adoptive Cell Transfer with TCRP1A CD8+T Cells

For the adoptive cell transfer (ACT), P1A-specific (TCRP1A) CD8+T cells were isolated from spleens and lymph nodes of TCRP1A mice as described hereinabove, and stimulated in vitro by co-culture with irradiated (10,000 rads) L1210.P1A.B7-1 cells (Gajewski et al., 1995, J Immunol 154, 5637-5648) at 1:2 ratio (0.5×10⁵ CD8+T cells and 10⁵ L1210.P1A.B7-1 cells per well in 48-well plates) in IMDM (GIBCO®) containing 10% fetal bovine serum supplemented with L-arginine (0.55 mM, Merck®), L-asparagine (0.24 mM, Merck®), glutamine (1.5 mM, Merck®), beta-mercaptoethanol (50 μM, Sigma Aldrich®), 50 U mL⁻¹ penicillin and 50 mg mL⁻¹ streptomycin (Life Technologies®). Four days later, TCRP1A CD8+T cells were purified on a Lymphoprep gradient (StemCell®) and 10⁷ living cells were injected intravenously in 200 μL PBS in TiRP-tumor bearing mice on the day of randomization.

6—Evaluation of Alpha-2 Adrenaline Receptor Agonists/Antagonists on Tumor Growth

a) TiRP Tumor Model

TiRP-tumor bearing mice received adoptive cell transfer (ACT) of 10⁷ P1A-specific activated CD8+T cells and daily injections of vehicle control, clonidine (5 mg/kg, i.p.), or clonidine (5 mg/kg, i.p.) combined with phentolamine (5 mg/kg, i.p.), when the tumor size was around 500 mm³, until the day of sacrifice. Tumor size was monitored every 2 days. Mice were sacrificed when the tumor in control group reached around 2,000 mm³.

b) CT26 Tumor Model

C57BL/6 mice were injected subcutaneously with 2×10⁶ CT26 colon tumor cells at an age of 6 to 8 weeks. For evaluation of phentolamine on the effect of clonidine and guanabenz, the mice were randomized into different groups when tumors size arrived around 50 mm³ and received daily injections of guanabenz (5 mg/kg, i.p.), a mix of guanabenz (5 mg/kg, i.p.) and phentolamine (5 mg/kg, i.p.), clonidine (5 mg/kg, i.p.), a mix of clonidine (5 mg/kg, i.p.) and phentolamine (5 mg/kg, i.p.), a mix of clonidine (5 mg/kg, i.p.) and propranolol (5 mg/kg, i.p.) or vehicle control. For evaluation of yohimbine on the effect of clonidine and guanabenz, the mice were randomized into different groups when tumors size arrived around 100 mm³ and received daily injections of guanabenz (5 mg/kg, i.p.), yohimbine (10 mg/kg), a mix of guanabenz (5 mg/kg, i.p.) and yohimbine (10 mg/kg, i.p.), clonidine (5 mg/kg, i.p.), a mix of clonidine (5 mg/kg, i.p.) and yohimbine (10 mg/kg, i.p.) or vehicle control. Tumor size was measured every 2 days and mice were sacrificed when the tumor in control group reached 1,000 mm³.

### Example 2

### Colon Carcinoma Model

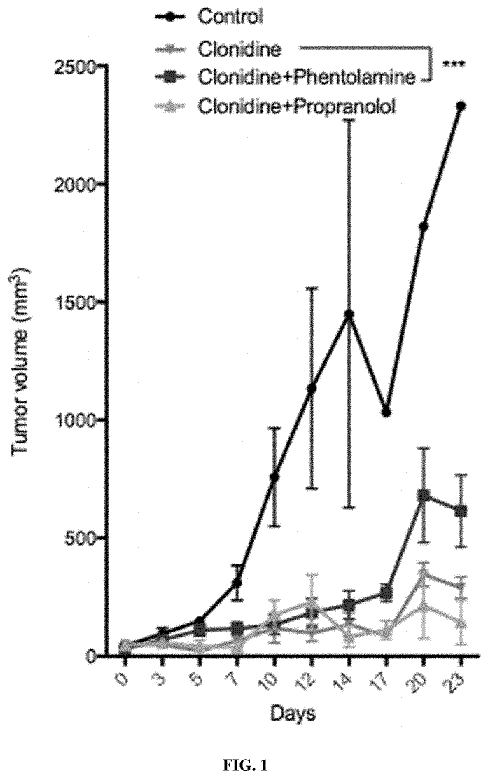

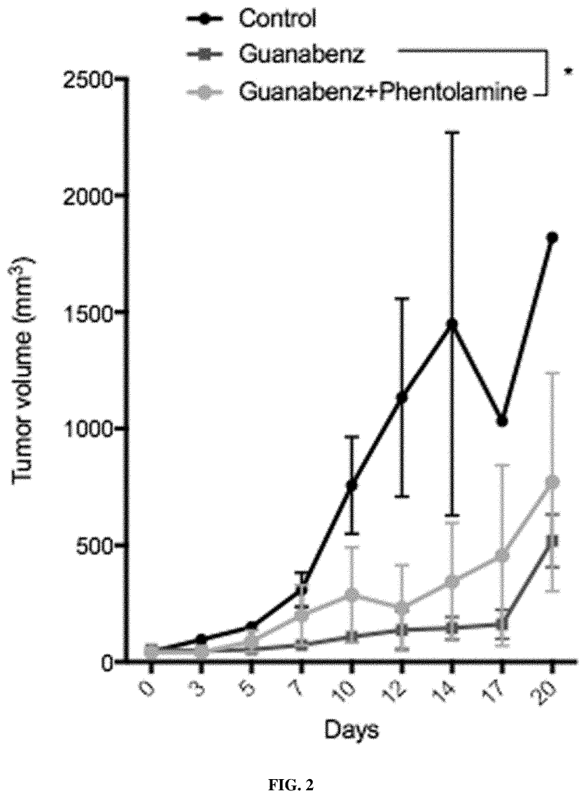

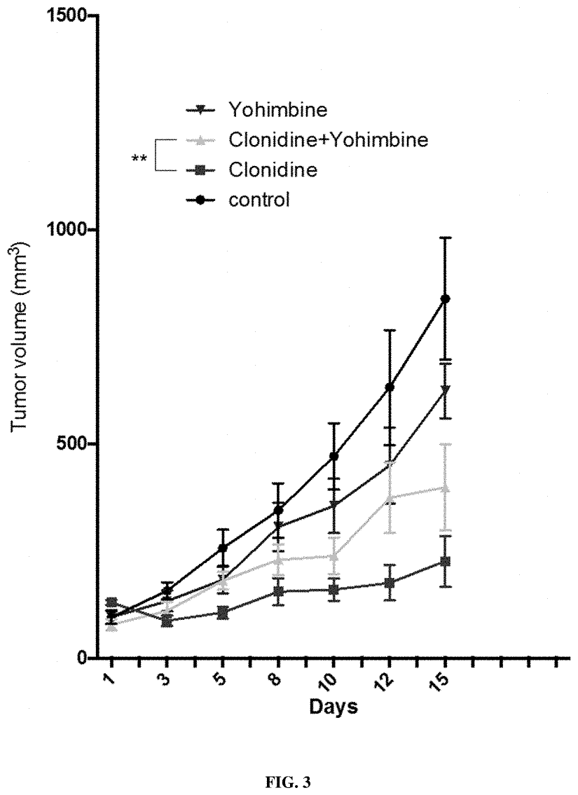

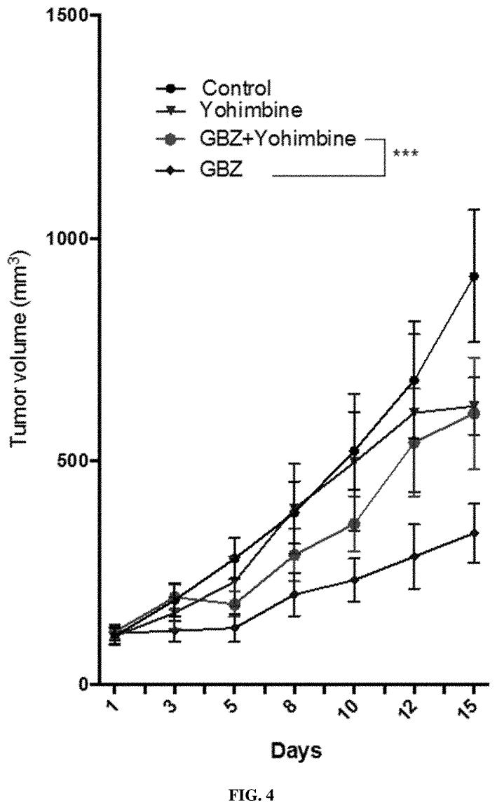

BALB/c mice bearing CT26 colon carcinomas received daily injections of vehicle control, clonidine (5 mg/kg, i.p.), clonidine (5 mg/kg, i.p.) plus phentolamine (5 mg/kg, i.p.), or clonidine (5 mg/kg, i.p.) plus propranolol (5 mg/kg, i.p.), when the tumor size was around 50 mm³, until the day of sacrifice. As shown on FIG. 1 and FIG. 2, respectively, clonidine and guanabenz strongly inhibited CT26 tumor growth. Phentolamine, an alpha-2 adrenergic receptor antagonist, attenuated the inhibitory effect of clonidine and guanabenz on tumor growth, indicating that inhibition of tumor growth is mediated via the agonistic effect on alpha-2 adrenergic receptor. In contrast, propranolol, a beta-adrenergic receptor antagonist, did not revert the effect of clonidine on tumor growth (FIG. 2). The anti-tumor effect of clonidine and guanabenz through the agonistic effect on alpha-2 adrenergic receptor was further confirmed by the fact that yohimbine, an alpha-2 adrenergic receptor antagonist, also reduced the inhibition of tumor growth (see FIG. 3 and FIG. 4).

### Example 3

### Romifidine and Clonidine Administration Promote an Increase of Tumor Infiltration of CD8+T Cells

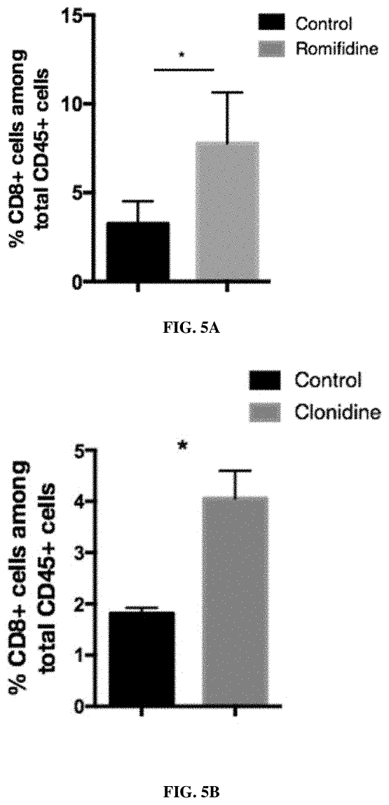

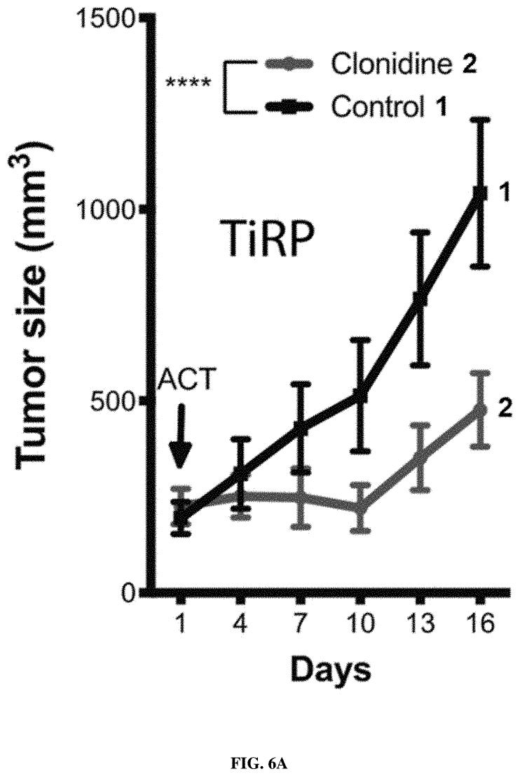

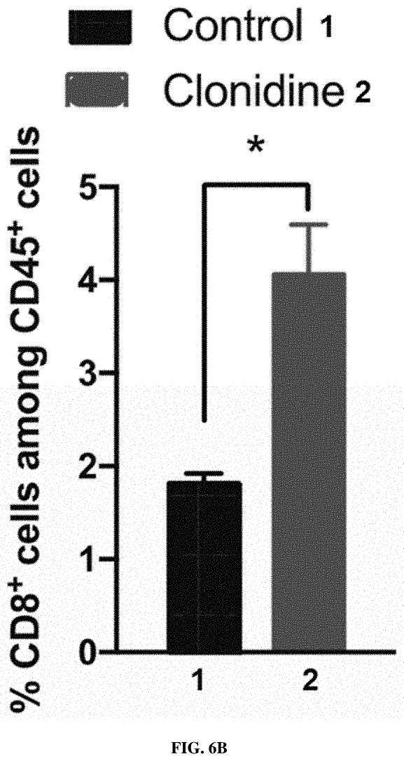

TiRP-tumor bearing mice received adoptive cell transfer (ACT) of 10⁷ of P1A-specific activated CD8+T cells. Daily injections of 5 mg/kg i.p. of romifidine or clonidine were thus administered from the day of the ACT, when the tumor size was around 500 mm³, until the day of sacrifice. As shown on FIGS. 5A-B, following romifidine or clonidine administration, tumor infiltration of CD8+T cells was increased. These results demonstrate that alpha-2 adrenergic receptor agonists, when combined with adoptive T cell transfer, efficiently promote an anticancer response. As shown on FIGS. 6A-B, clonidine, when combined with ACT, efficiently control the growth of immune-resistant autochthonous melanoma tumors (TiRP).

### Example 4

### Effect of the Combined Treatment of Clonidine and ACT Towards Human Ovarian Tumor

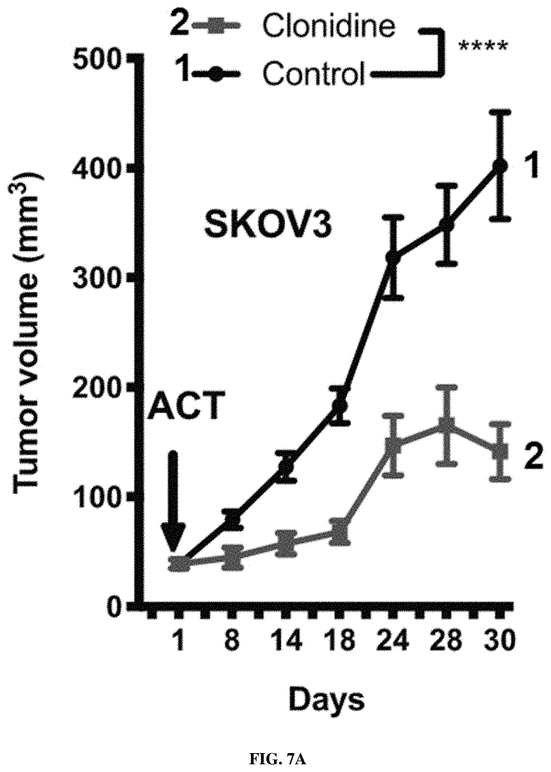

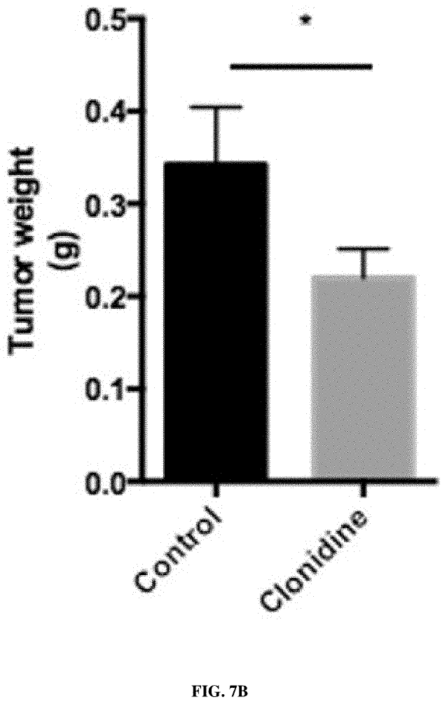

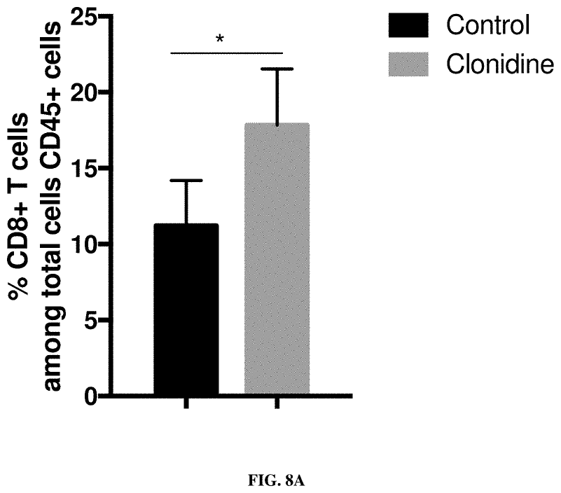

SKOV3 human ovarian tumor cells were injected subcutaneously in immune deficient NSG mice. When tumors were palpable, tumor-bearing mice received adoptive cell transfer of 3×10⁶ of allogeneic human PBMC (i.v.) and were injected daily with clonidine (5 mg/kg, i.p.) or vehicle (PBS, i.p.) until sacrifice. As shown on FIG. 7, clonidine strongly increased the anti-tumor efficacy of human PBMC against human ovarian carcinoma cells SKOV3. Both tumor growth (FIG. 7A) and tumor weight on the day of sacrifice (FIG. 7B) were significantly decreased with clonidine used with an ACT, as compared to ACT alone. These results demonstrate that clonidine improves the therapeutic efficacy of human T cells in a human xenograft model, and that the therapeutic effect of clonidine could be translated to human tumors.

### Example 5

### Clonidine Improves the Therapeutic Efficacy of Human T Cells in a Human Xenograft Model

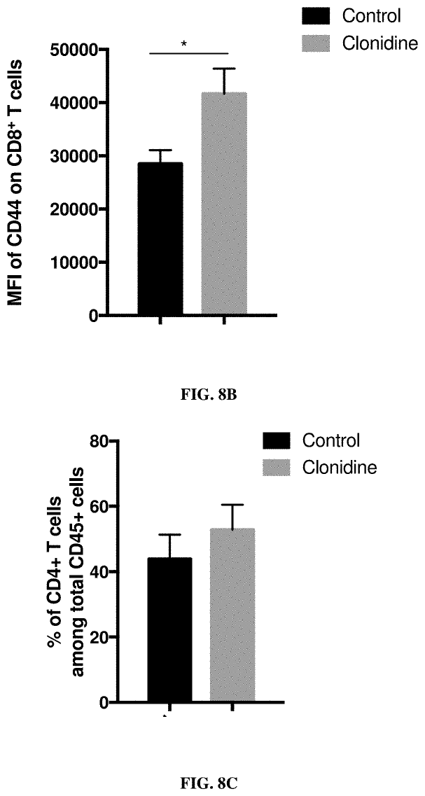

SKOV3 human ovarian tumor cells were injected subcutaneously in immune deficient NSG mice. When tumors were palpable, tumor-bearing mice received adoptive cell transfer of 3×10⁶ of allogeneic human PBMC (i.v.) and were injected daily with clonidine (5 mg/kg, i.p.) or vehicle (PBS, i.p.) until sacrifice. Following clonidine administration (5 mg/kg, i.p.), infiltration of both CD8+(FIG. 8A) and CD4+(FIG. 8C) human T cells into human ovarian tumors SKOV3 was increased, and the tumor-infiltrated CD8+T cells were also more active in the mice that received clonidine, as shown with the increased expression of CD44 on tumor-infiltrated CD8+T cells (FIG. 8B).

### Example 6

### Clonidine Strongly Sensitized Immune-Resistant Autochthonous Melanoma Tumors (TiRP) to Adoptive Cell Transfer (ACT)

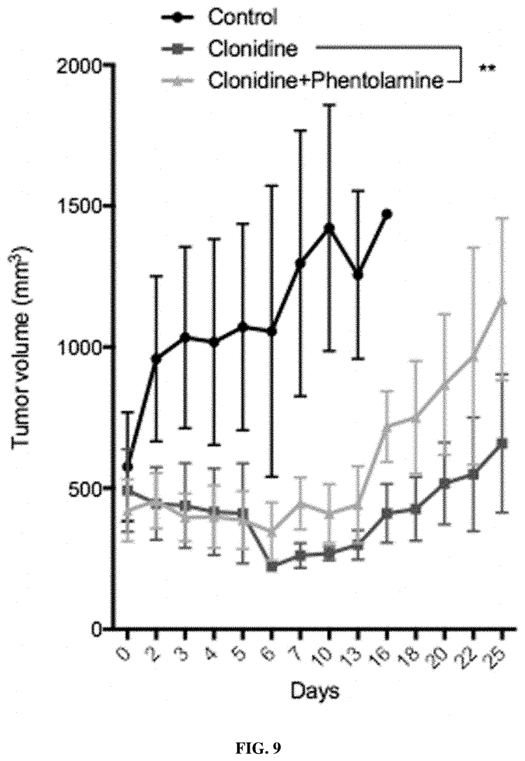

TiRP-tumor bearing mice received adoptive cell transfer (ACT) of 10⁷ P1A-specific activated CD8+T cells and daily injections of vehicle control, clonidine (5 mg/kg, i.p.), or clonidine (5 mg/kg, i.p.) plus phentolamine (5 mg/kg, i.p.), when the tumor size was around 500 mm³, until the day of sacrifice. As shown on FIG. 9, clonidine strongly sensitized immune-resistant autochthonous melanoma tumors (TiRP) to adoptive cell transfer (ACT). Phentolamine attenuated the inhibitory effect of clonidine on TiRP tumor growth.

### Example 7

### Both Clonidine and Guanfacine Treatment as a Monotherapy Result in an Immune-Mediated Tumor Inhibition of Melanoma

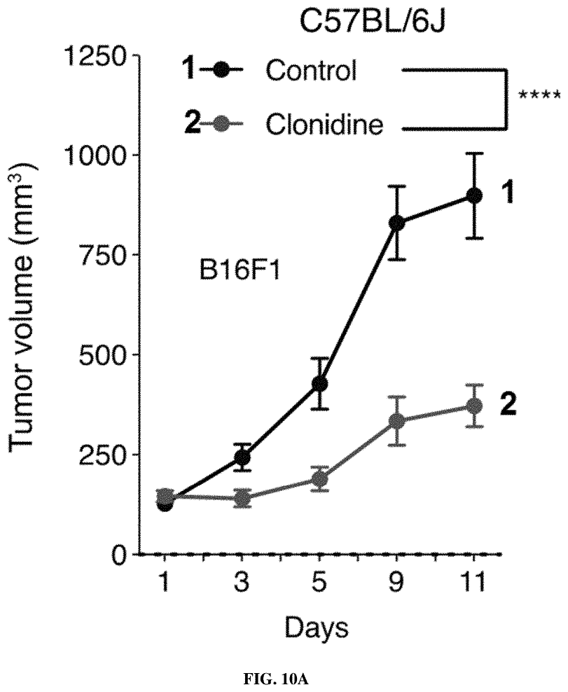

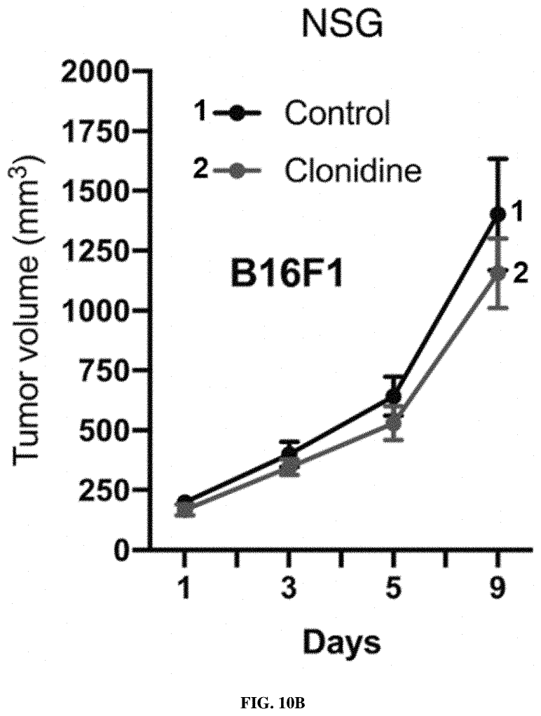

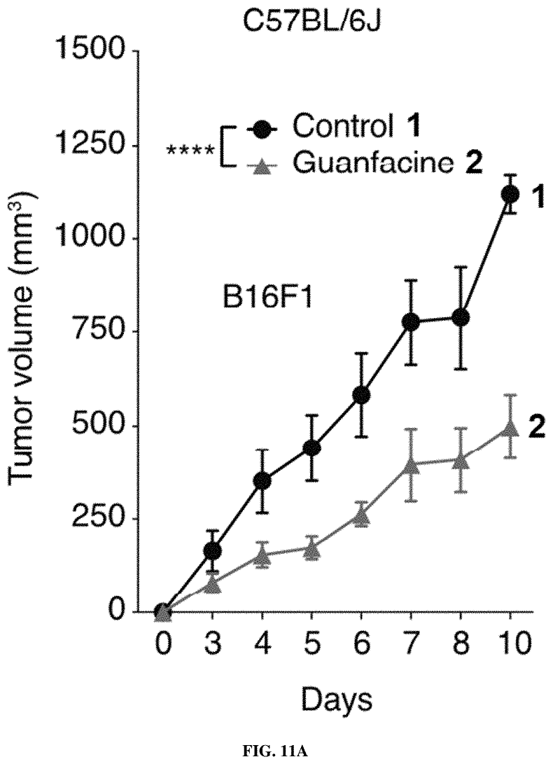

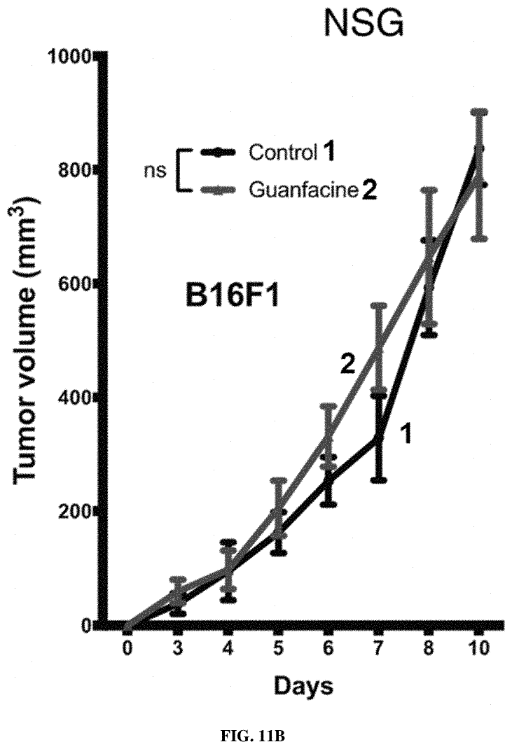

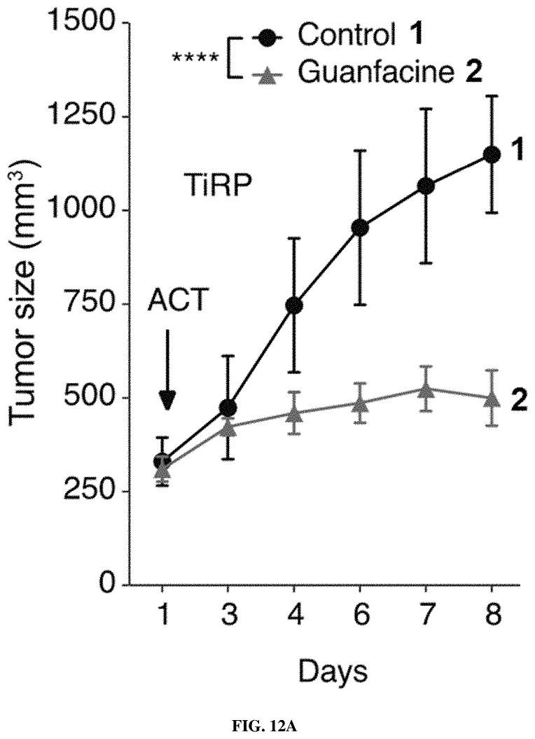

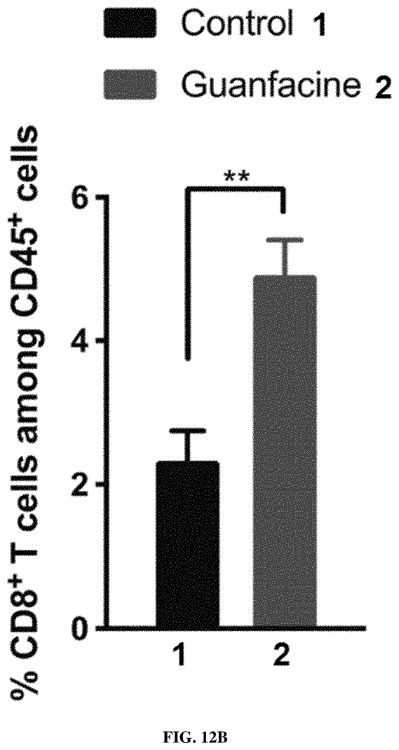

Treatments of B16F1 melanoma with either clonidine (FIGS. 10A-B) or guanfacine (FIGS. 11A-B) inhibited tumor growth when B16F1 was implanted in immune competent C57BL/6 mice. No tumor inhibition was found when B16F1 was implanted in immune deficient NSG Mice. Both clonidine and guanfacine inhibited the tumor growth in the induced TiRP model (FIGS. 6A-B and FIGS. 12A-B) despite the fact that this model is extremely immune suppressive.

### Example 8

### Anti-Tumor Efficiency of Clonidine or Guanabenz as Monotherapy in the Myelofibrosis (Blood Cancer)

Myelofibrosis (MF) is a clonal malignant disease resulting from acquisition of JAK-STAT activating driver mutations in bone marrow cells, of which the JAK2V617F mutation is the most prevalent.

Bone marrow from primary CD45.2 JAK2V617F mice was mixed 1:1 with CD45.1 C57BL/6 marrow and transplanted into lethally irradiated CD45.2 C57BL/6 recipients. 1 month after bone marrow transplantation, mice were randomized into three groups and received daily i.p injection of 5 mg/kg of clonidine, guanabenz or PBS. Blood was taken on Day 0 (Just before the treatment) and on Day 7 (7 days after the treatment). The allele burden was assessed as a fraction of CD45.2 cells along total CD45 cells in the blood of mice.

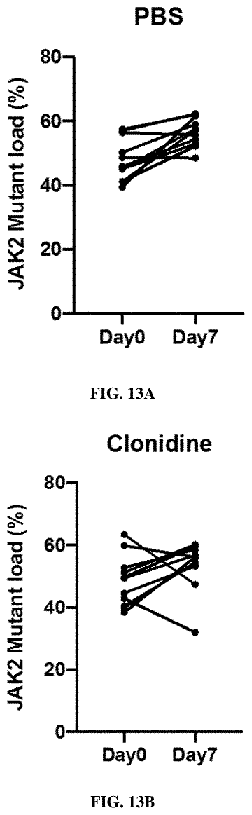

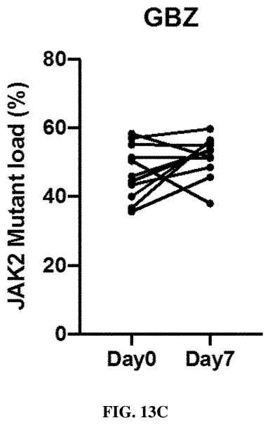

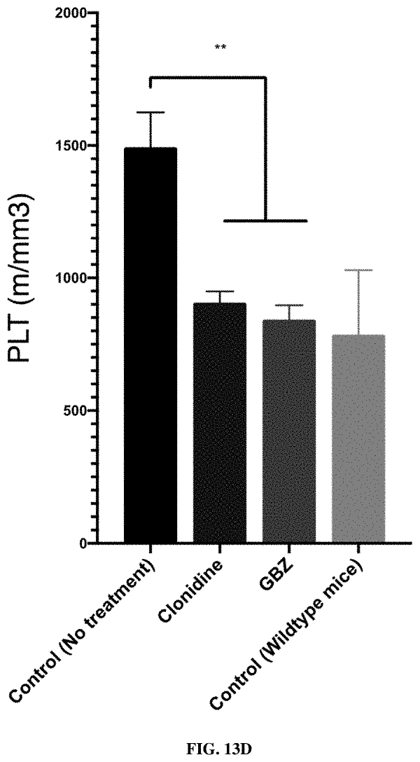

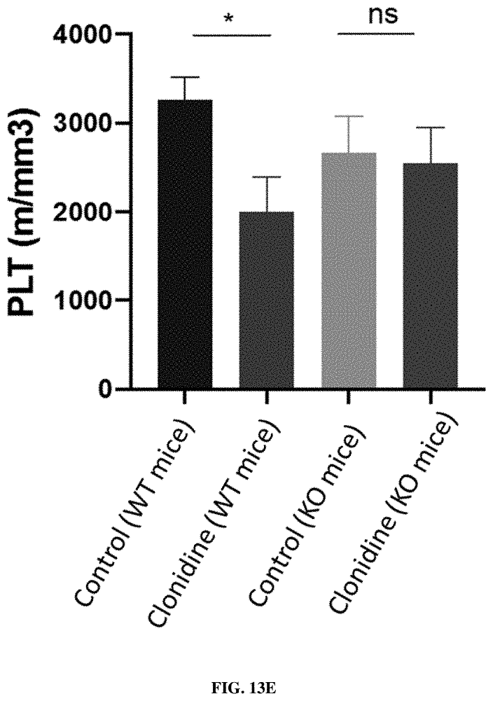

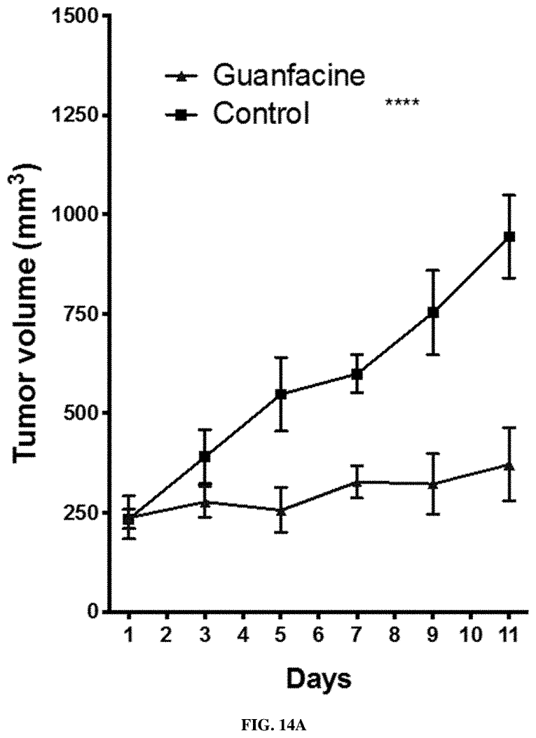

Decreased JAK2V617F allele burden is a golden standard method to evaluate the disease progression of MF. As shown in FIG. 13A, in mice that did not receive treatment (PBS group), the JAK2V617F allele burden continues to increase as expected. In mice treated with clonidine (FIG. 13B) or guanabenz (FIG. 13C), 1 week after treatment, there is a decrease of JAK2V617F allele burden in a number of mice.

Murine femurs and tibias from JAK2V617F mutant mice were first harvested and cleaned thoroughly. Marrow cells were flushed into PBS with 2% fetal bovine serum using a 25G needle and syringe. Resulting cell suspensions were then filtered through a 40 uM cell strainer. Recipient mice were irradiated with two doses of 500 rad 4 h apart. 1 million of donor cells were injected into wild-type recipients by standard intravenous injection using a 27G insulin syringe. One week after bone marrow transplantation, mice were treated with guanabenz or clonidine at a dose of 5 mg/kg daily for three weeks. After three weeks of treatment, mice were dissected and peripheral blood was drawn by cardiac puncture, the platelet concentration was measured by Cell Counter Analyzer MS9-5V. Wildtype mice that did not receive bone marrow transplantation or clonidine/guanabenz treatment were used as controls. As shown in FIG. 13D, both clonidine and guanabenz treatments significantly reduced the high platelet counts induced by JAK2V617F mutated bone marrow.

Romiplostim is a ligand binding to the Thrombopoietin Receptor (TPO) Ligand. It has been shown that thrombopoietin (TPO)/myeloproliferative leukemia protein (MPL; TPO receptor) signaling pathway plays a certain role in the development of MF. Myeloproliferative leukemia protein activation directly induces fibrocyte differentiation to cause myelofibrosis. Administration of TPO ligand induced MF.

8-week female C57BL/6 J wild-type and Adra2a KO mice were administered saline or 1 mg/kg of romiplostim by a subcutaneous injection into the neck skin on days 1, 8 and 15. Clonidine or guanabenz were administrated i.p. at a dose of 5 mg/kg daily for two weeks starting from day 8. Mice were dissected on day 22 and peripheral blood was drawn by cardiac puncture, the platelet concentration was measured by Cell Counter Analyzer MS9-5V.

As shown in FIG. 13E, TPO activation increases the platelet counts in the blood, which is another phenotype of MF. Clonidine treatment reduces the platelet count in the wildtype mice but not in the KO mice, indicating that alpha-2 adrenergic receptor activation inhibits the increase of platelet related to the disease progression.

### Example 9

### Anti-Tumor Efficiency of Guanfacine or Clonidine as Monotherapy in the TiRP Melanoma Model

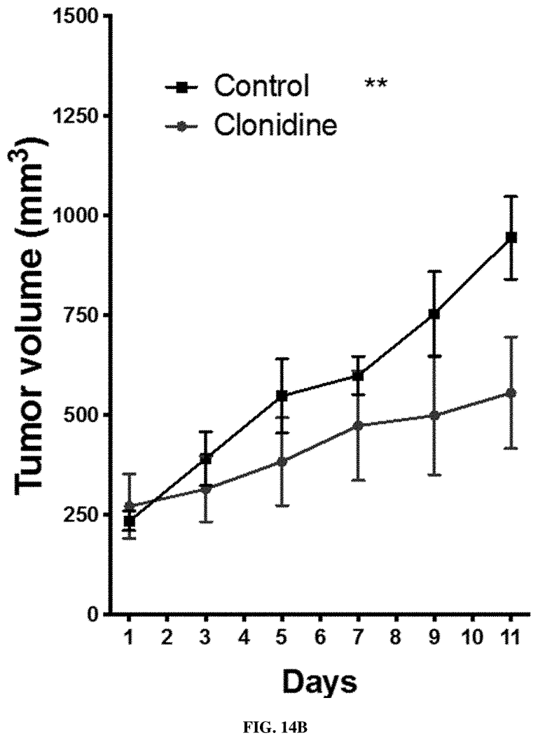

FIGS. 14A-B shows that both guanfacine (FIG. 14A) and clonidine (FIG. 14B) are efficient in decreasing the tumor volume in a TiRP melanoma model, when administered as a monotherapy.

### Example 10

### Anti-Tumor Efficiency of Clonidine or Guanabenz Treatment Towards Colon Cancer is Blocked by Yohimbine, an Alpha-2 Adrenergic Receptor Antagonist

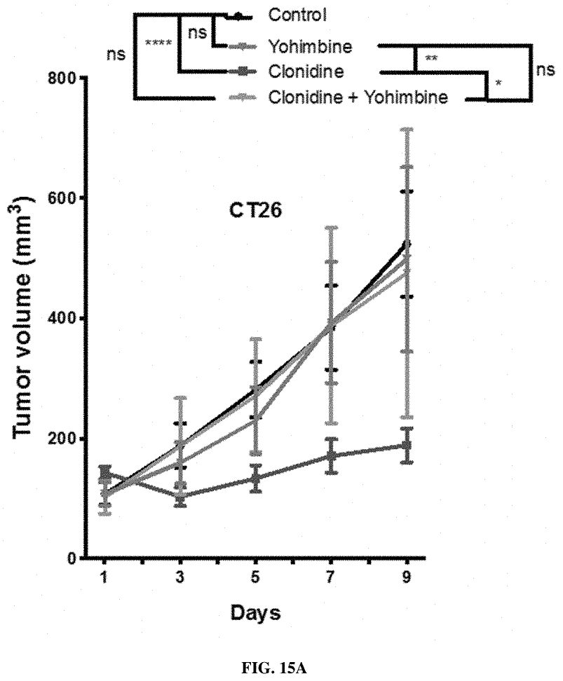

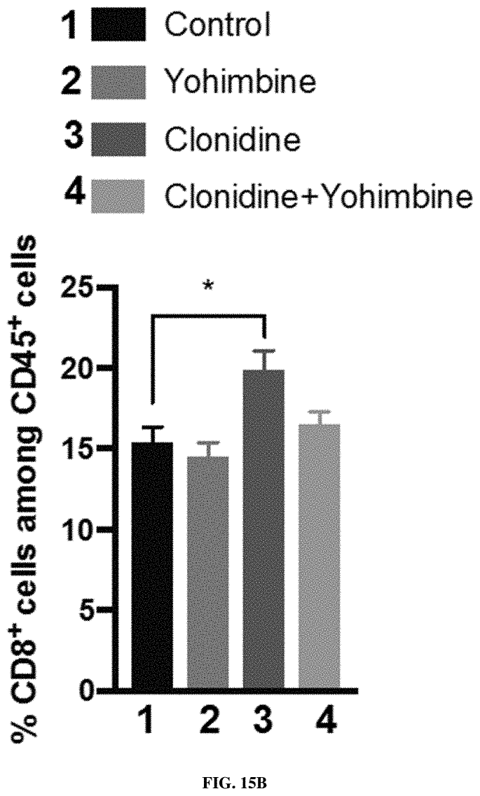

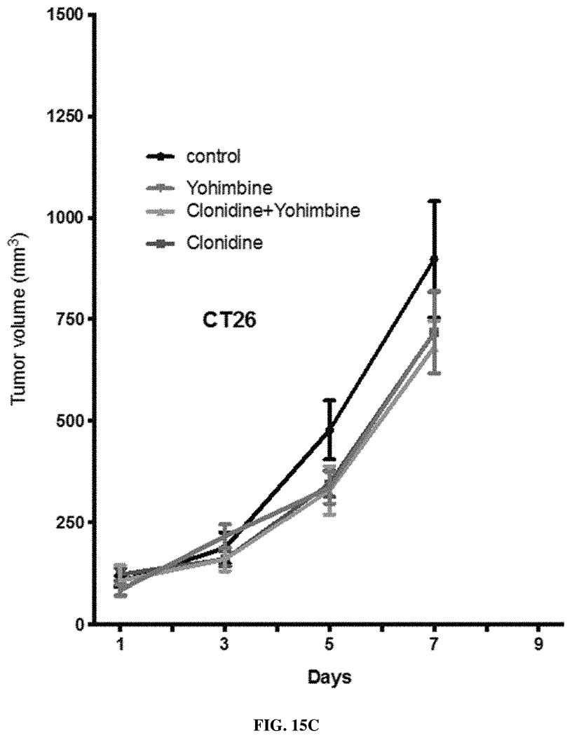

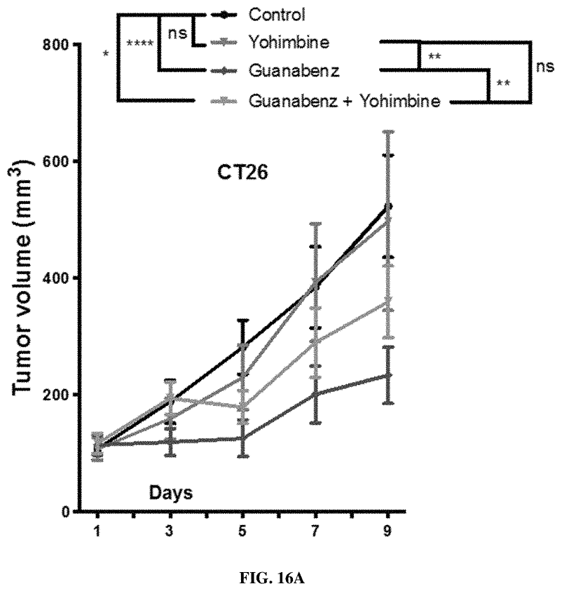

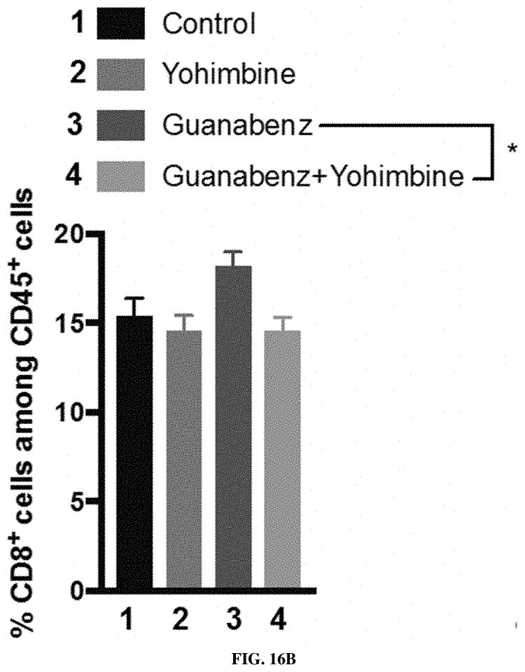

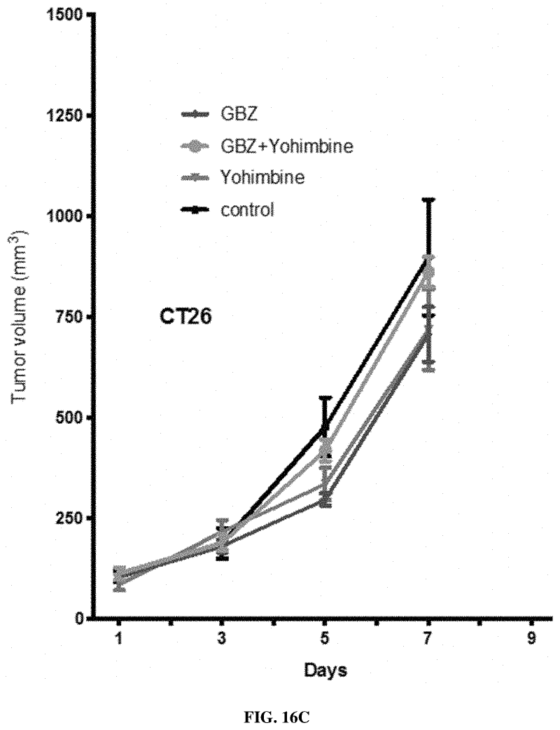

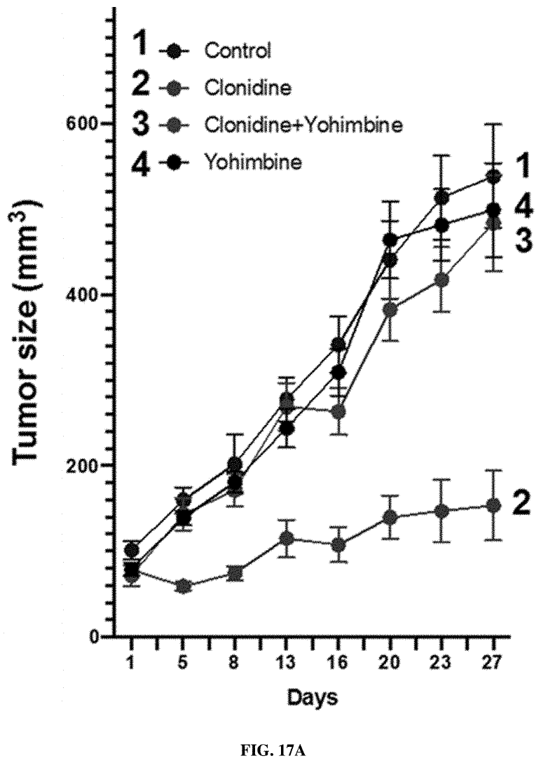

Clonidine treatment (FIGS. 15A-C) or guanabenz treatment (FIGS. 16A-C) inhibited CT26 colon cancer tumor growth, and these effects were strongly attenuated when combined with yohimbine, an alpha-2 adrenergic receptor antagonist, indicating both clonidine and guanabenz inhibit CT26 tumor growth via their agonistic effect on alpha 2 adrenergic receptor.

### Example 11

### Anti-Tumor Efficiency of Clonidine or Guanabenz Treatment Towards Human Ovarian Cancer is Blocked by Yohimbine, an Alpha-2 Adrenergic Receptor Antagonist

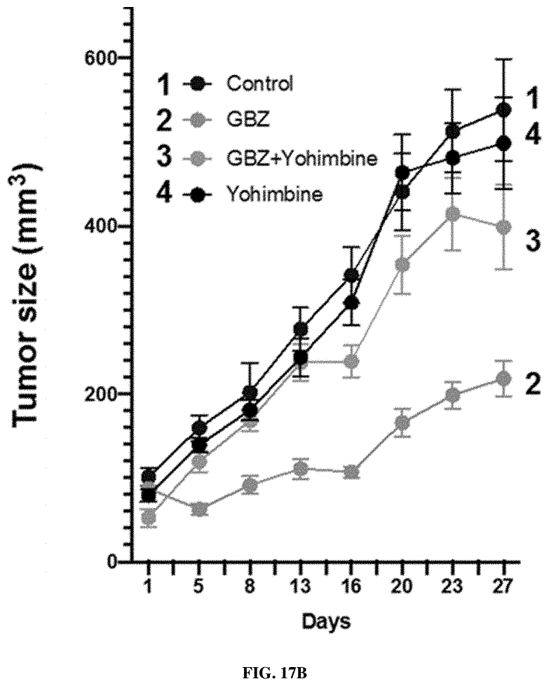

Clonidine treatment (FIG. 17A) or guanabenz treatment (FIG. 17B) resulted in a significant tumor inhibition in human LS411N ovarian cancer xenograft tumor model upon adoptive transfer of human allogenic PBMC, therefore indicating the therapeutic effect observed in the murine tumor models could be translated to human tumors. This effect was strongly attenuated when combined with yohimbine, an alpha-2 adrenergic receptor antagonist, indicating that also for human tumors, the mechanism of action for both clonidine and guanabenz is via their agonistic effect on alpha-2 adrenergic receptor.

### Example 12

### Anti-Tumor Efficiency of Clonidine or Guanabenz Treatment Towards Colon Carcinoma is Abolished in Adra2a KO Mice

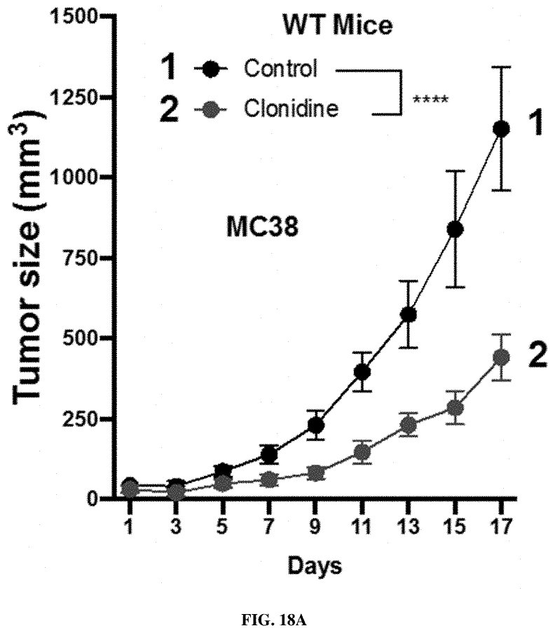

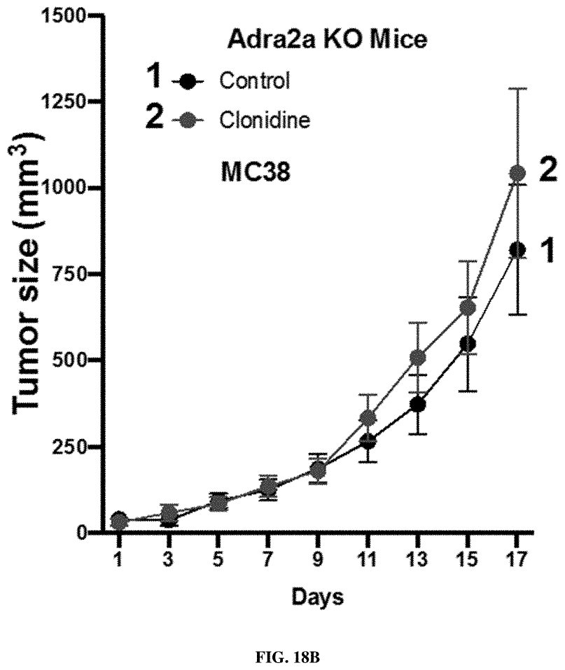

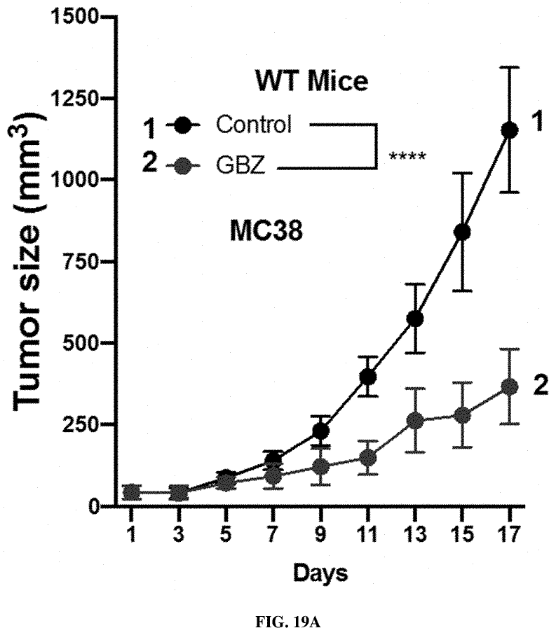

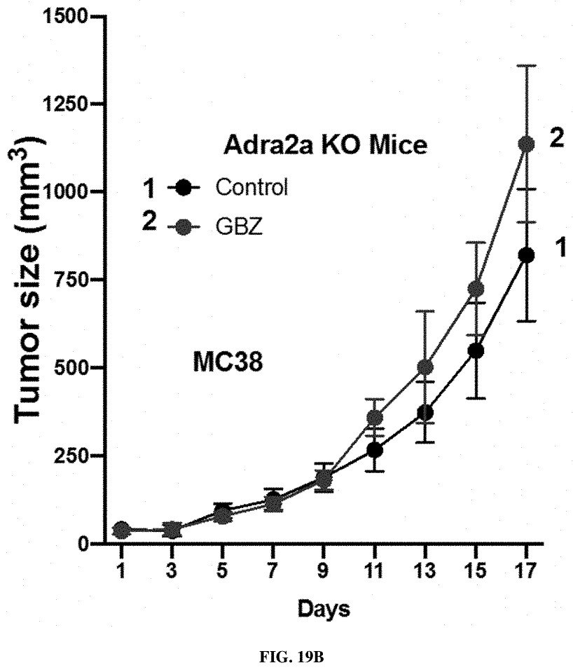

C57BL/6 Wildtype or C57BL/6 Adra2a KO mice bearing MC38 colon carcinomas received daily injections of vehicle control (PBS), clonidine (5 mg/kg, i.p.) or guanabenz (5 mg/kg, i.p.), when the tumor size was around 50 mm³, until the day of sacrifice. In the wildtype mice, in consistence with previous observations, a significant tumor inhibition was observed both with clonidine treatment (FIG. 18A) and guanabenz treatment (FIG. 19A). However, this tumor inhibition was almost completely attenuated in Adra2a KO mice (FIG. 18B and FIG. 19B). These results provided evidences that alpha-2 adrenergic receptor is the valid immunotherapeutic target which inhibition can promote anti-tumor immune response.

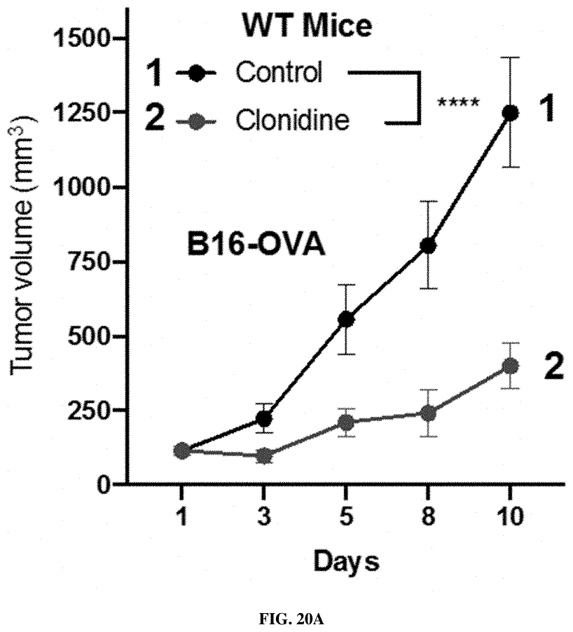

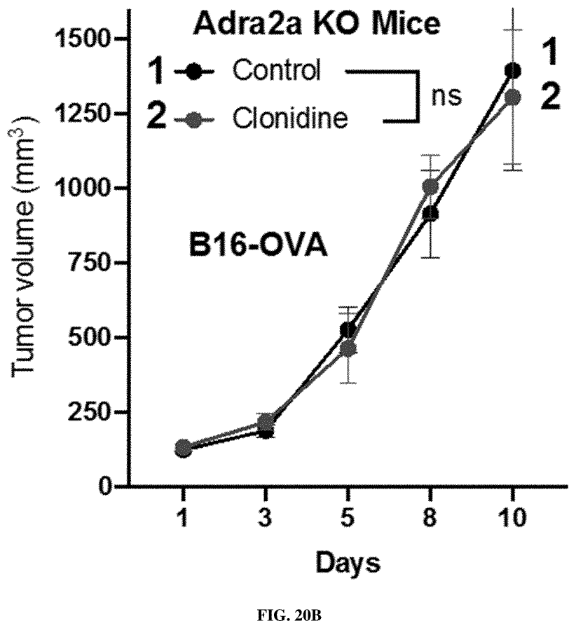

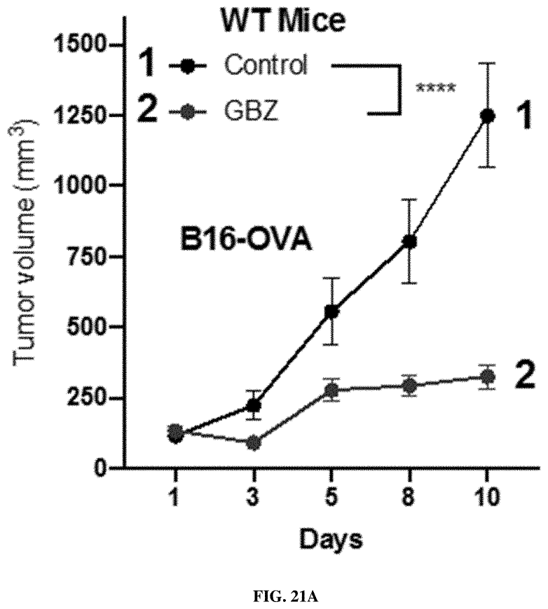

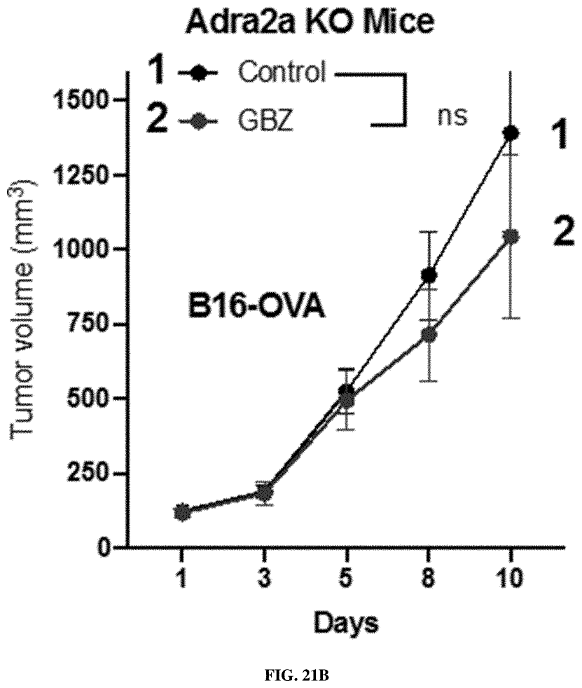

Similar observation was made for C57BL/6 Wildtype (FIG. 20A and FIG. 21A) or C57BL/6 Adra2a KO (FIG. 20B and FIG. 21B) mice bearing B16F10 OVA melanoma.

### Example 13

### Anti-Tumor Efficiency of a Clonidine and Guanabenz Treatments Towards Various Cancer Types

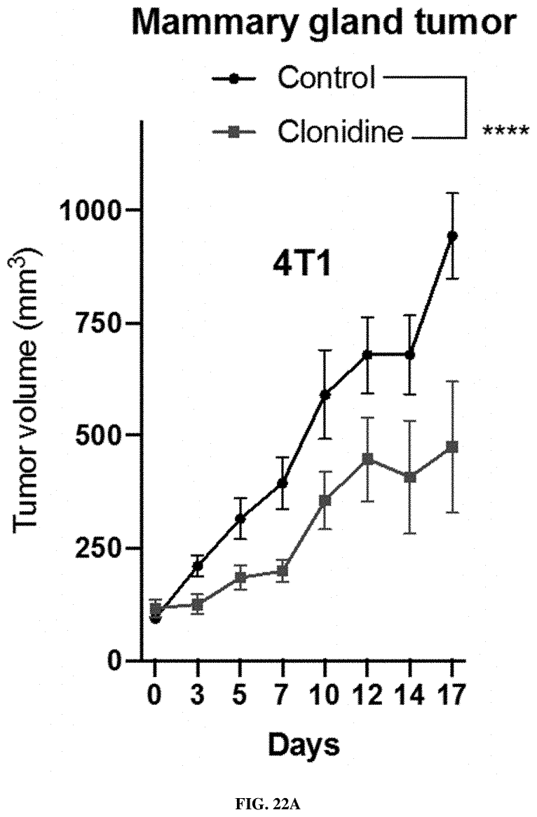

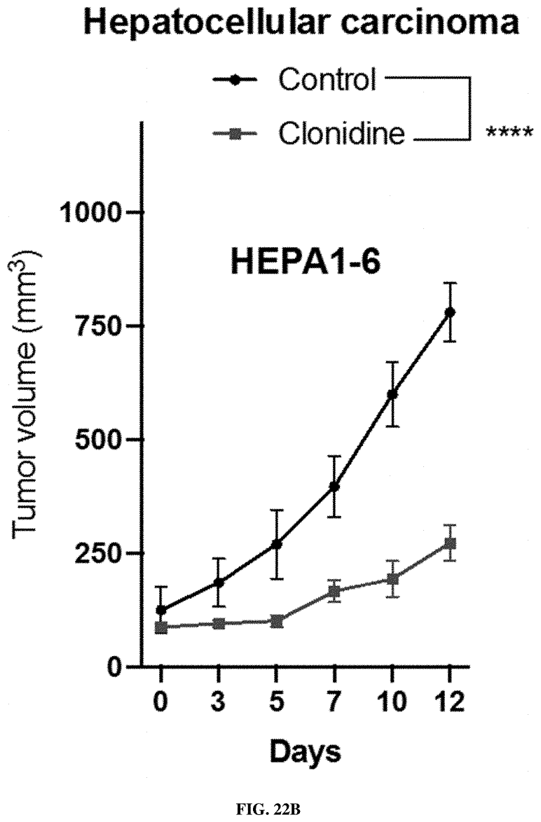

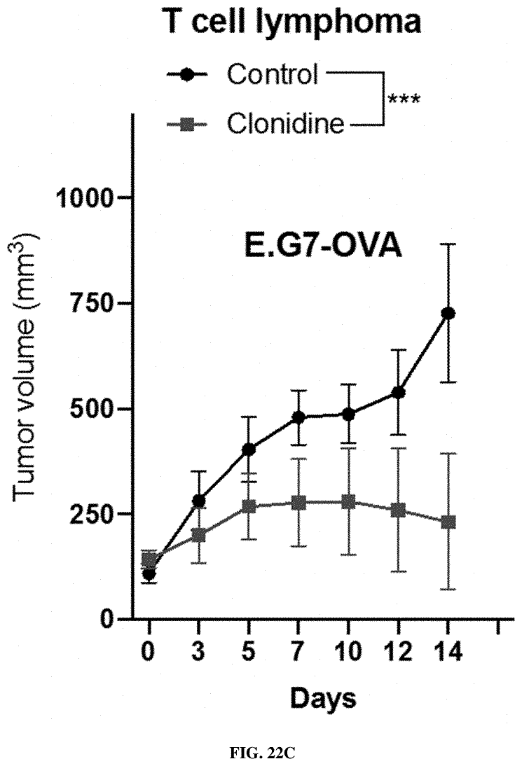

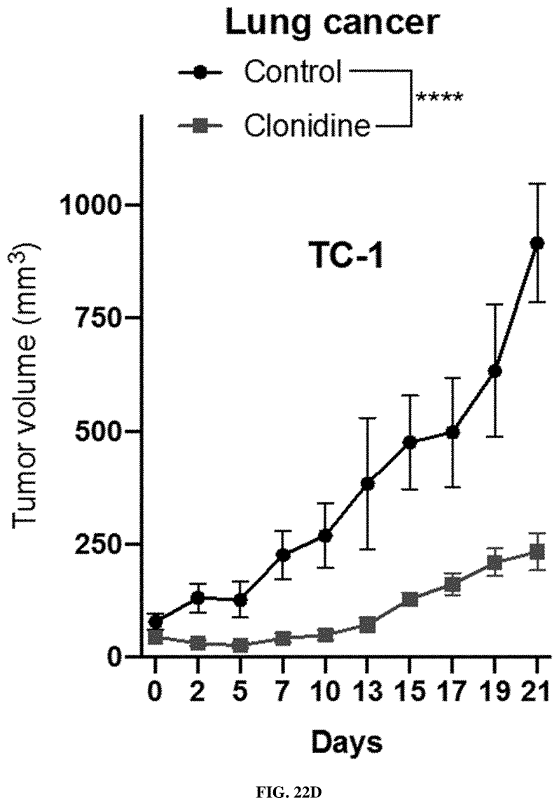

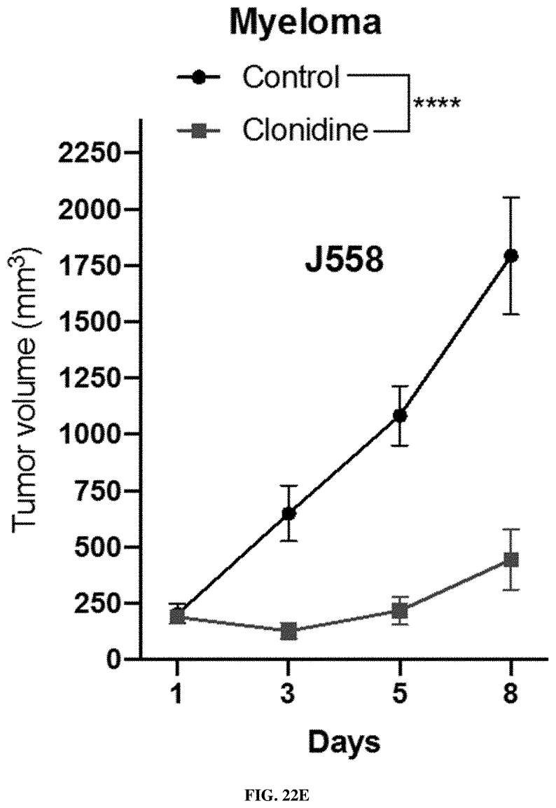

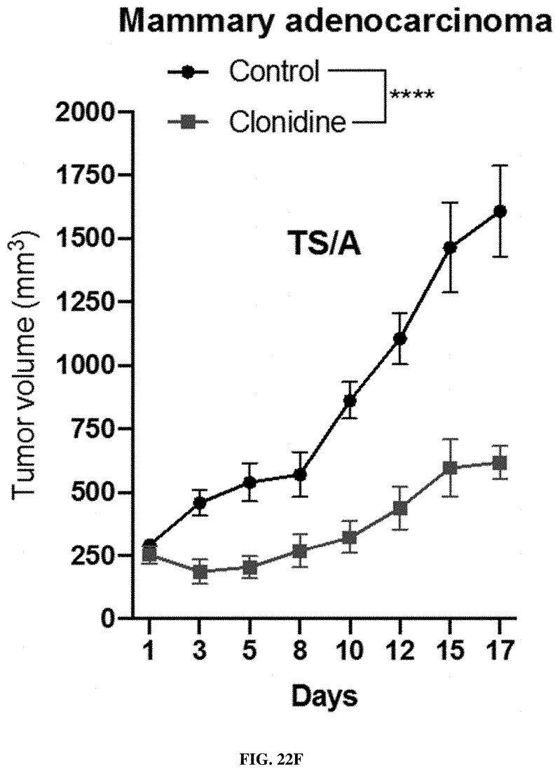

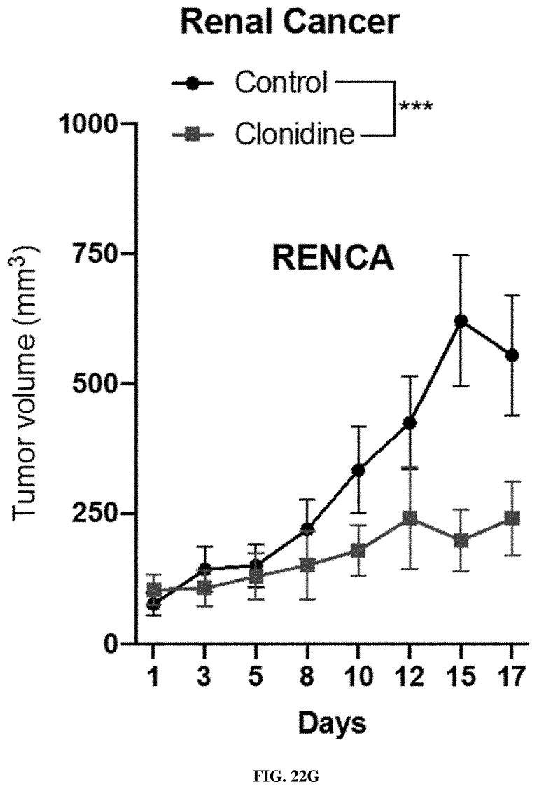

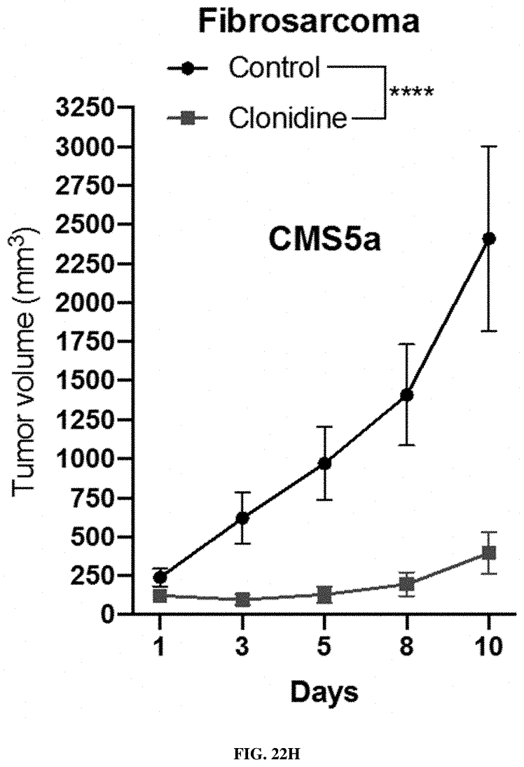

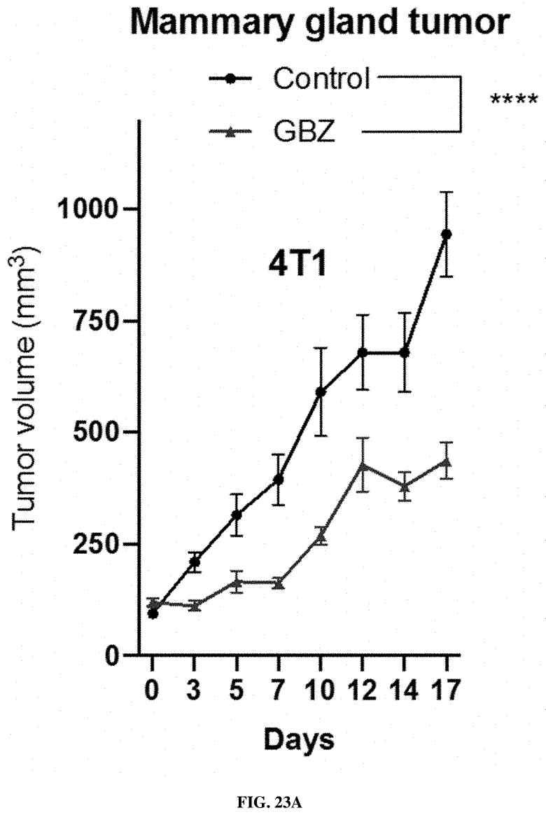

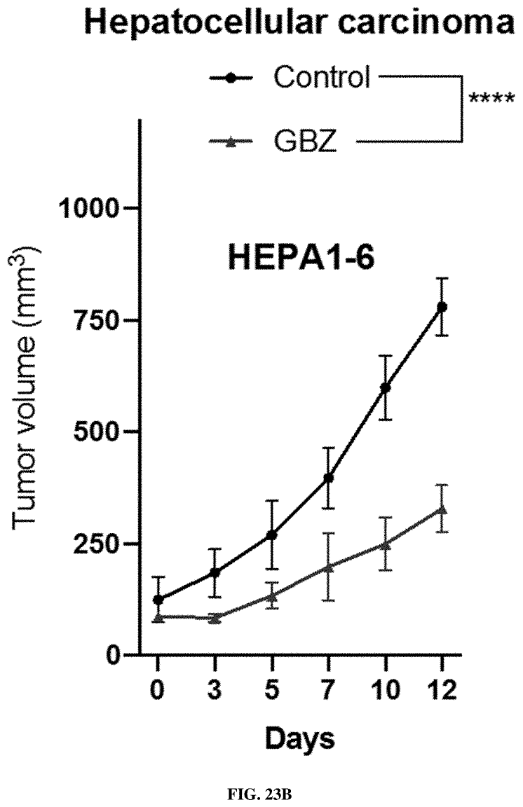

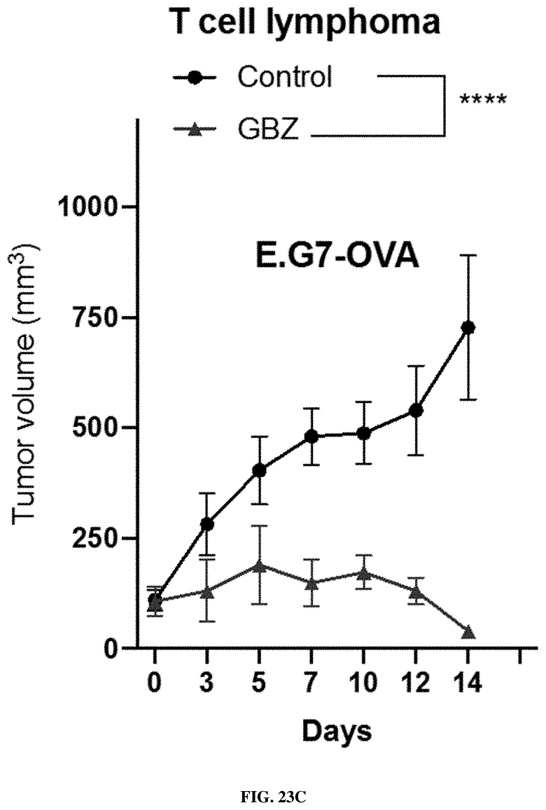

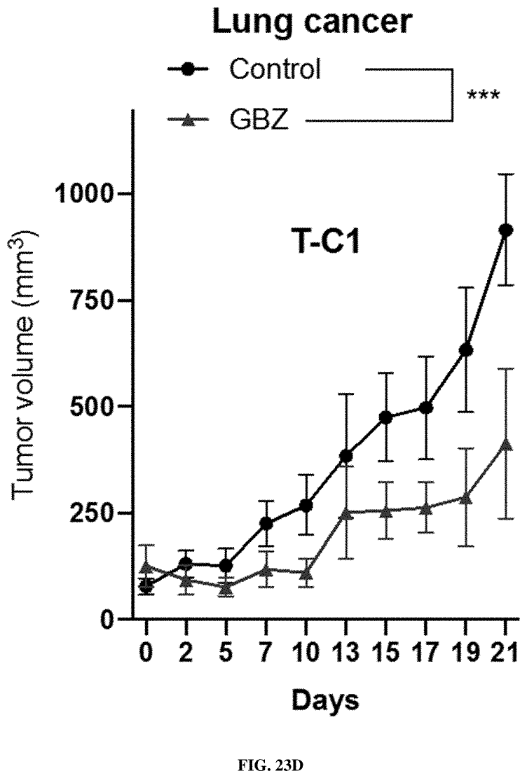

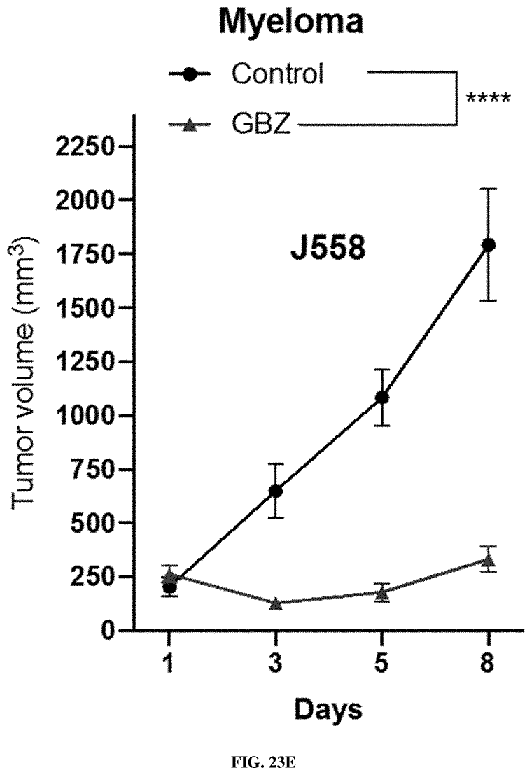

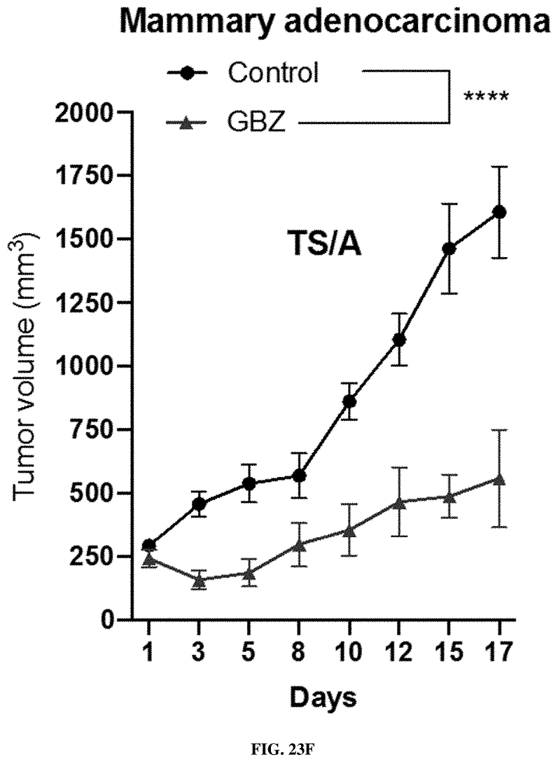

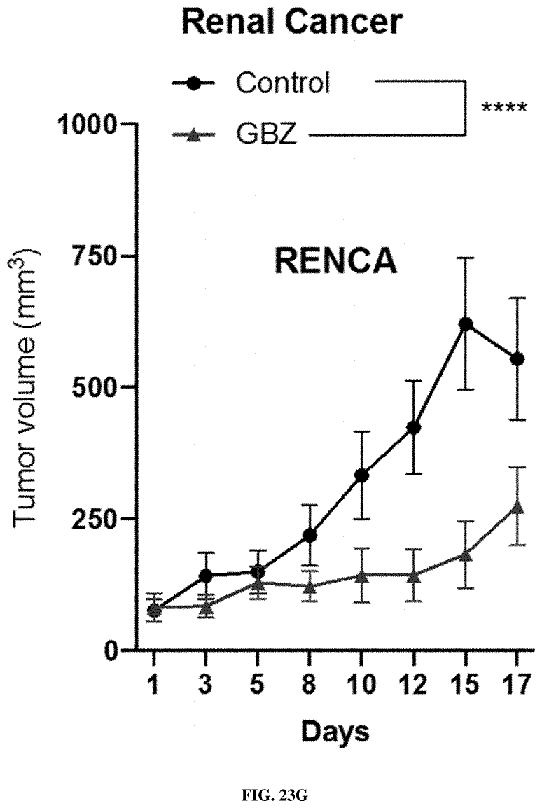

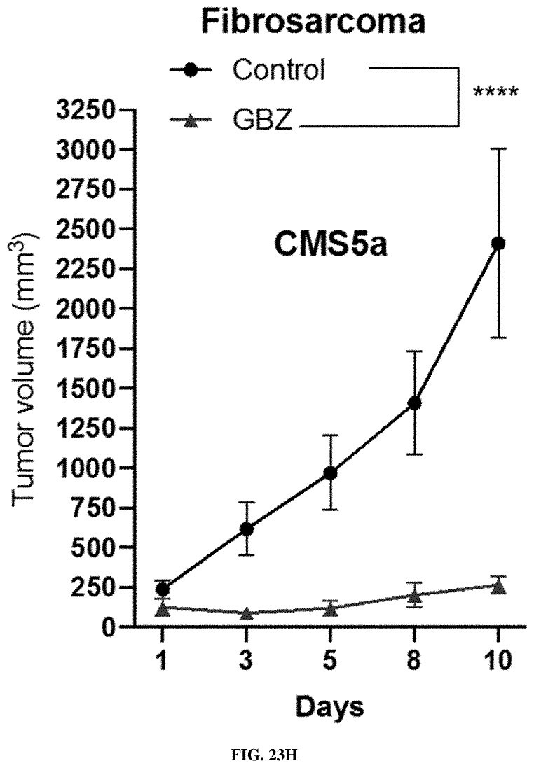

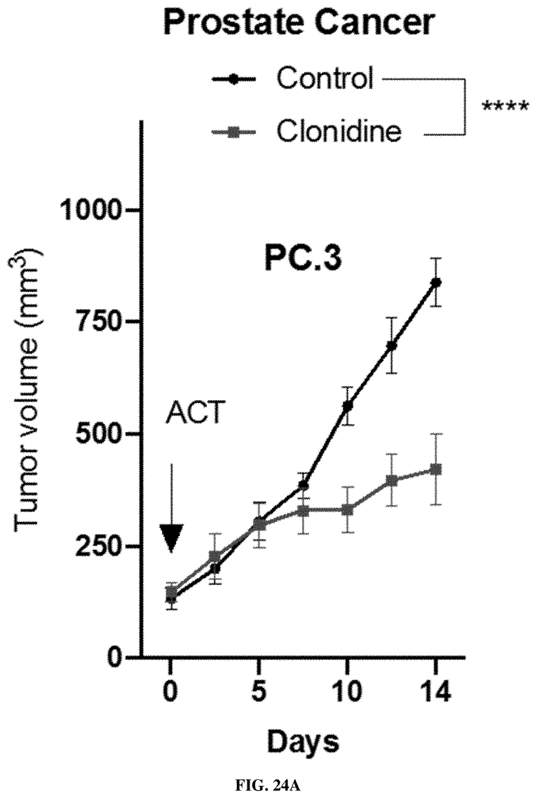

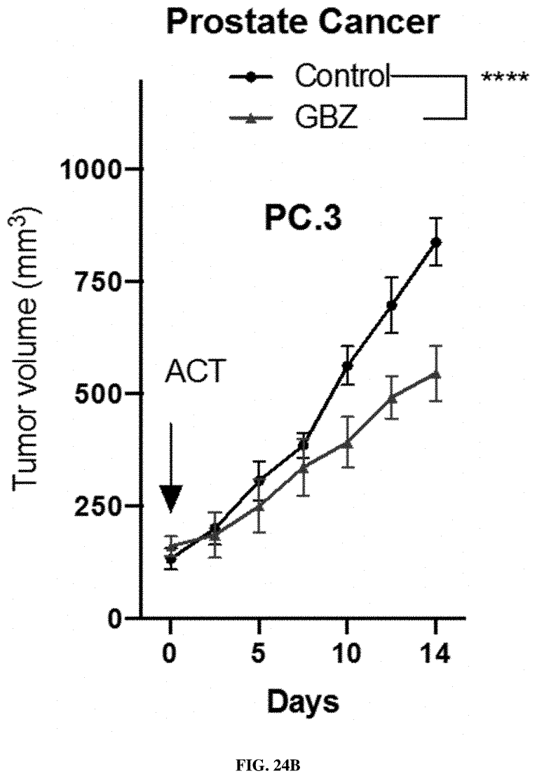

Clonidine treatment led to a strong tumor inhibition in various tumor models, such as mammary gland tumor (FIG. 22A), hepatocellular carcinoma (FIG. 22B), T cell lymphoma (FIG. 22C), lung cancer (FIG. 22D), myeloma (FIG. 22E), mammary adenocarcinoma (FIG. 22F), renal cancer (FIG. 22G) and fibrosarcoma (FIG. 22H). The same observations were made for guanabenz treatment (FIGS. 23A-H). Finally, clonidine or guanabenz treatment led to a strong tumor inhibition in a xenograft model of prostate cancer in NSG mice treated with PBMC (see FIGS. 24A-B).

Altogether, these results are suggesting that adrenergic receptor alpha-2 agonists can be used to treat a broad range of cancer types.

### Example 14

### Anti-Tumor of Apraclonidine Treatment Towards Colon Cancer is Mediated by Peripheral Rather than Nervous Central Effects

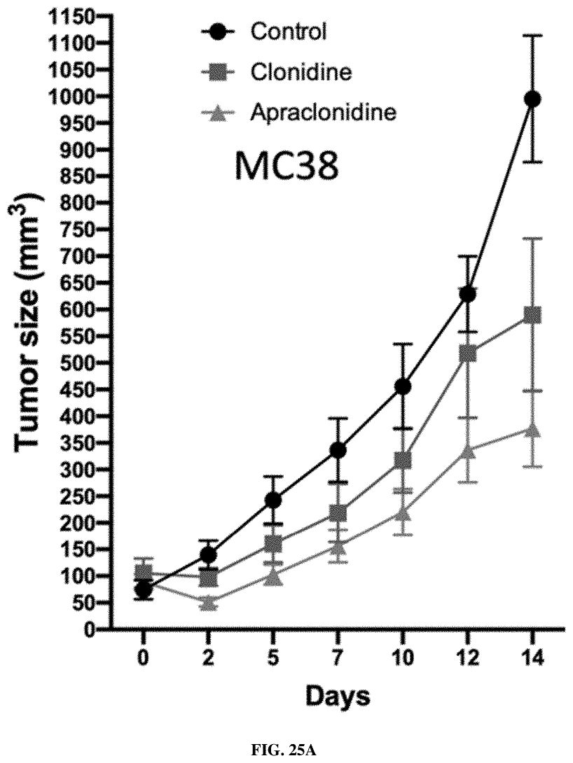

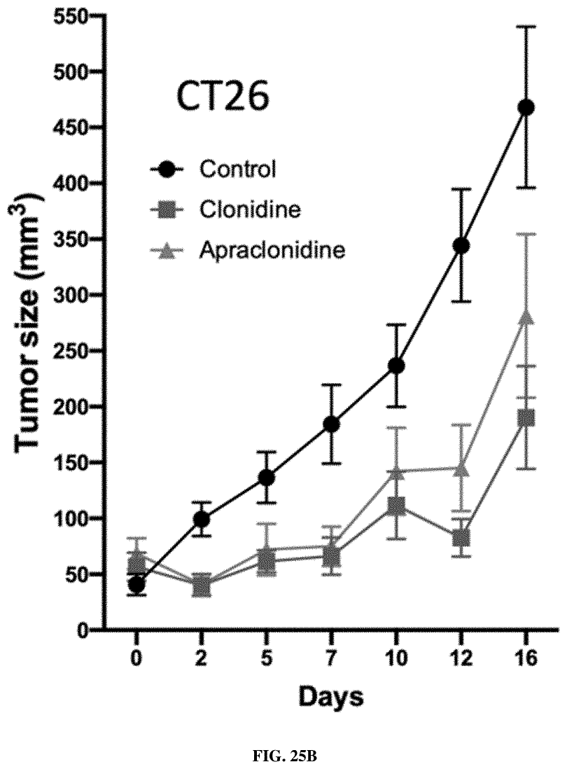

Apraclonidine does not cross the blood-brain barrier as compared to clonidine. FIGS. 25A-B show that tumor growth of MC38 colon cancer (FIG. 25A) or CT26 colon cancer (FIG. 25B) treated with PBS, clonidine or apraclonidine. The anti-tumor effect of apraclonidine suggests that the pharmacological profile of alpha-2 adrenergic receptor agonists may be explained by peripheral effects rather than nervous central effects.

1-15. (canceled)

16. A method for the prevention and/or the treatment of cancer in an individual in need thereof comprising the administration of a therapeutic effective amount of an alpha-2 adrenergic receptor agonist, as an active agent.

17. The method according to claim 16, wherein said agonist is selected from the group consisting of amitraz, apraclonidine, bethanidine, brimonidine, bromocriptine, cirazoline, clonidine, detomidine, dexmedetomidine, dipivefrin, droxidopa, epinephrine, ergotamine, etilefrine, etomidate, fadolmidine, guanabenz, guanfacine, guanoxabenz, guanethidine, indanidine, lofexidine, medetomidine, mephentermine, metamfetamine, metaraminol, methoxamine, dl-methylephedrine, methyldopa, mivazerol, moxonidine, naphazoline, norepinephrine, norfenefrine, octopamine, oxymetazoline, pergolide, phenylpropanolamine, propylhexedrine, pseudoephedrine, racepinephrine, rilmenidine, romifidine, (R)-3-nitrobiphenyline, synephrine, talipexole, tizanidine, xylazine, xylometazoline, and a functional derivative thereof.

18. The method according to claim 16, wherein said agonist is selected from the group consisting of apraclonidine, clonidine, guanfacine, romifidine, and a functional derivative thereof.

19. The method according to claim 16, wherein said agonist is selected from the group consisting of an antibody, an antibody fragment, an afucosylated antibody, a diabody, a triabody, a tetrabody, a nanobody, and an analog thereof.

20. The method according to claim 16, wherein said agonist does not cross the blood/brain barrier.

21. The method according to claim 16, wherein said agonist is to be administered at a dose ranging from about 0.0001 mg/kg body weight to about 100 mg/kg body weight.

22. The method according to claim 16, wherein said agonist is to be administered systemically.

23. The method according to claim 16, wherein said agonist is to be administered with an additional treatment selected from the group consisting of chemotherapy, immunotherapy, radiation, and the like.

24. The method according to claim 16, wherein said cancer is selected from the group consisting of myelofibrosis, acute lymphoblastic leukemia, acute myeloblastic leukemia adrenal gland carcinoma, bile duct cancer, bladder cancer, breast cancer, cervical cancer, colorectal cancer, endometrial cancer, esophageal cancer, gastric cancer, gastrointestinal stromal tumors, glioblastoma, head and neck cancer, hepatocellular carcinoma, Hodgkin's lymphoma, kidney cancer, lung cancer, melanoma, Merkel cell skin cancer, mesothelioma, multiple myeloma, myeloproliferative disorders, non-Hodgkin lymphoma, ovarian cancer, pancreatic cancer, prostate cancer, salivary gland cancer, sarcoma, squamous cell carcinoma, testicular cancer, thyroid cancer, urothelial carcinoma, and uveal melanoma.

25. The method according to claim 16, wherein the cancer is a solid cancer.

26. The method according to claim 25, wherein the solid cancer is selected from the group consisting of melanoma, breast carcinoma, colon carcinoma, renal carcinoma, adrenocortical carcinoma, testicular teratoma, skin sarcoma, fibrosarcoma, lung carcinoma, adenocarcinoma, liver carcinoma, fibrosarcoma, glioblastoma, prostate carcinoma, ovarian cancer and pancreatic carcinoma.

27. The method according to claim 25, wherein the solid cancer is selected in the group consisting of colon carcinoma, ovarian cancer, melanoma, breast carcinoma, liver carcinoma, lung carcinoma, renal carcinoma, prostate carcinoma and fibrosarcoma.

28. A method for reducing the volume and/or the weight of a solid tumor in an individual in need thereof comprising the administration of a therapeutic effective amount of an alpha-2 adrenergic receptor agonist, as an active agent.

29. The method according to claim 28, wherein said agonist is selected from the group consisting of apraclonidine, clonidine, guanfacine, romifidine, and a functional derivative thereof.

30. The method according to claim 28, wherein said agonist is selected from the group consisting of an antibody, an antibody fragment, an afucosylated antibody, a diabody, a triabody, a tetrabody, a nanobody, and an analog thereof.

31. The method according to claim 28, wherein said agonist does not cross the blood/brain barrier.

32. A kit for preventing and/or treating cancer comprising:
an alpha-2 adrenergic receptor agonist or a pharmaceutical composition comprising the same, and
a means to administer the alpha-2 adrenergic receptor agonist or the pharmaceutical composition.

33. The kit according to claim 32, further comprising an anti-tumor compound.

34. The kit according to claim 32, wherein the anti-tumor compound is selected from the group consisting of a chemotherapy agent and an immunotherapy agent.
